SEQUENCE LISTING (16 sequences)

SEQ ID NO: 1 - protein, 105 aa, Kunjin virus
MSKKPGGPGKSRAVNMLKRGMPRVLSLTGLKRAMLSLIDGRGPTRFVLALLAFFRFTAIAPTRAVLDRWRSVNKQTAMKHLLSFKKELGTLTSAINRRSSKQKKR

SEQ ID NO: 2 - protein, 127 aa, Japanese encephalitis virus
MTKKPGGPGKNRAINMLKRGLPRVFPLVGVKRVVMSLLDGRGPVRFVLALITFFKFTALAPTKALLGRWRAVEKSVAMKHLTSFKRELGTLIDAVNKRGKKQNKRGGNERSIMWLASLAIVTAYAGA

SEQ ID NO: 3 - protein, 127 aa, Japanese encephalitis virus
MTKKPGGPGKNRAINMLKRGLPRVFPLVGVKRVVMSLLDGRGPVRFVLALITFFKFTALAPTKALLGRWKAVEKSVAMKHLTSFKRELGTLIDAVNKRGRKQNKRGGNEGPIMWLASLAVVIACAGA

SEQ ID NO: 4 - protein, 112 aa, Tick-borne encephalitis virus
MARKAILKGKGGGPPRRVSKETAKKTRQSRVQMPNGLVLMRMMGILWHAIAGTARSPVLKSFWNSVPLKQATAALRKIKKAVSTLMVGLQRRGKKRSTTDWMGWLLVTVLLG

SEQ ID NO: 5 - DNA, 24 bp, Artificial Sequence
  WNV 5′ Primer
accatgtctaagaaaccaggaggc

SEQ ID NO: 6 - DNA, 24 bp, Artificial Sequence
  WNV 3′ Primer
tgctcctacgctggcgatcaggcc

SEQ ID NO: 7 - DNA, 21 bp, Artificial Sequence
  CK2-alpha 5′ primer
atgtcgggacccgtgccaagc

SEQ ID NO: 8 - DNA, 24 bp, Artificial Sequence
  CK2-alpha 3′ primer
ttactgctgagcgccagcggcagc

SEQ ID NO: 9 - DNA, 24 bp, Artificial Sequence
  CK2-beta 5′ primer
atgagcagctcagaggaggtgtcc

SEQ ID NO: 10 - DNA, 27 bp, Artificial Sequence
  CK2-beta 3′ primer
tcagcgaatcgtcttgactgggctctt

SEQ ID NO: 11 - protein, 8 aa, West Nile virus
MLSLIDGK

SEQ ID NO: 12 - protein, 8 aa, Artificial Sequence
  WNV c protein peptide
MLALIDGK

SEQ ID NO: 13 - protein, 19 aa, West Nile virus
SKKPGGPGKSRAVNMLKRG

SEQ ID NO: 14 - protein, 19 aa, West Nile virus
KRAMLSLIDGKGPIRFVLA

SEQ ID NO: 15 - protein, 19 aa, West Nile virus
TLTSAINRRSSKQKKRGGK

SEQ ID NO: 16 - protein, 9 aa, Artificial Sequence
  negative contorl peptide
RREEETEEE

(19) United States
(12) Patent Application Publication
(10) Pub. No.: US 2010/0256217 A1
(43) Pub. Date: Oct. 7, 2010

(54) ANTIVIRAL INHIBITION OF CASEIN KINASE II

(75) Inventors: David Weiner, Merion, PA (US); Mathura Ramanathan, Ardmore, PA (US)

Correspondence Address: Pepper Hamilton LLP, 400 Berwyn Park, 899 Cassatt Road, Berwyn, PA, 19312-1183, US

(73) Assignee: The Trustees of the University of Pennsylvania, Philadelphia, PA (US)

(21) Appl. No.: 12/301,993

(22) PCT Filed: May 22, 2007
(86) PCT No.: PCT/US2007/069472

§ 371 Date: Sep. 25, 2009

Related U.S. Application Data
(60) Provisional application No. 60/802,828, filed on May 22, 2006.
(60) Provisional application No. 60/879,766, filed on Jan. 9, 2007.

(51) Int. Cl. A61K 31/7088; A61K 31/352; A61K 31/122; A61P 31/12; C12Q 1/70

(52) U.S. Cl. 514/44 A; 514/456; 514/680; 435/5

(57) ABSTRACT

Method of treating an individual exposed to and/or infected with a virus selected from the group consisting of: West Nile Virus, Japanese encephalitis virus, Kunjin virus Tick-borne encephalitis virus and Hepatitis C virus, are disclosed. The methods comprise administering to such individuals, a therapeutically effective amount of one or more compounds that inhibit CK2 activity, one or more compounds that inhibit CK2 expression or a combination thereof. Pharmaceutical compositions comprising therapeutically effective amount of one or more compounds that inhibit CK2 activity, one or more compounds that inhibit CK2 expression, or a combination thereof are also disclosed. Methods of inhibiting viral replication by a virus selected from the group consisting of: West Nile Virus, Japanese encephalitis virus, Kunjin virus Tick-borne encephalitis virus and Hepatitis C virus, using one or more compounds that inhibit CK2 activity, one or more compounds that inhibit CK2 expression and combinations thereof, are disclosed. Methods of identifying compound useful to treat infection by a virus selected from group consisting of: West Nile Virus, Japanese encephalitis virus, Kunjin virus Tick-borne encephalitis virus and Hepatitis C virus, and methods of identifying CK2 inhibitors are disclosed.

The present application claims priority to U.S. provisional application Ser. Nos. 60/802,828, filed May 22, 2006 and 60/879,766, filed Jan. 9, 2007, each of which is incorporated herein by reference. The present invention relates to composition for and methods of inhibiting the activity or expression of casein kinase II in order to inhibit virion formation.

### FIELD OF THE INVENTION

### Background of the Invention

West Nile virus (WNV), an emerging pathogen, has spread across the continental United States into Canada and Mexico, beginning with its dramatic appearance in New York in 1999. More than 16000 documented cases of WNV infection has occurred during last three years. Though human infection is usually asymptomatic, the elderly as well as immunocompromised individuals progress more frequently to the most severe form of the WNV disease, which displays encephalitis or inflammation of the brain. WNV viral pathogenesis is not completely understood and specific therapies for WNV have not yet been approved for use in humans.

WNV belongs to the JEV serological group under Flaviviridae. Flaviviruses are spherical enveloped positive-stranded RNA viruses with a diameter of 50 nm that are composed of multiples of copies of only three different proteins, namely C (capsid), E (envelope) and preM (precursor membrane protein). The genomic RNA is approximately 11000 nucleotides long and functions as the sole viral mRNA in infected cells. Translation of the single long open reading frame gives rise to three structural and seven nonstructural viral proteins. Coordinated activities of cellular signalases/proteases and viral proteases result in the formation of individual active gene products through post/co-translational modification.

The intracellular assembly of flaviviruses is not precisely understood; it is believed to take place at the ER since the viral particles first become visible in this compartment of infected cells. The role of C in virus assembly is ill-defined and still a matter of speculation. C is first synthesized as a membrane associated protein (anchored C) and the final mature form is processed by cleavage by the viral NS2B/NS3 proteolytic complex. The high content of basic amino acids reveals its potential to interact with RNA in the process of viral core formation. A wealth of recent information concerning the structure of flaviviruses has yielded atomic or lower resolution (Cryo-EM) structures. Yet these data did not reveal a well-defined, ordered organization of the nucleo-capsid and no details of C-protein-RNA and C-protein—envelope proteins interaction are known. It has been suggested that the early stages of the viral assembly depended on the interaction of the core protein with genome RNA. Reports on the characteristics of authentic flavivirus capsid and capsid protein isolated from virions are limited. Recently studies from closely related flaviviruses reported that the capsid protein exist predominantly in the dimer form.

The clinical manifestations of WN virus infection are well defined, but the mechanism of WNV pathogenesis remains unclear. A recent report elegantly described how WNV crosses the brain-blood-barrier (BBB). Induction of apoptotic pathways, and perivascular inflammation and microgliosis in neuronal cells, activation of NFkB and upregulation of MHC-I molecules, and the elevation of inflammatory cytokines are some of the notable changes in the cellular machinery observed rapidly in response to WNV infection.

Among the structural genes, C differs from preM and E in several aspects. Besides being rich in basic amino acids, C appears to harbor frequent leucine repeats, RNA binding properties, transcription activation domains, potential phosphorylation sites, and more importantly a bi-partite nuclear targeting sequence. Only C, not preM or E has been shown to localize in the nucleus, though cytoplasmic occurrences also were noted. These unique features suggest that the protein C, besides a structural role in virions, has the potential to contribute certain additional non-structural functions, such as a role in host cell pathogenesis.

Post-translational modifications of viral proteins play an important role in regulating their activity, localization, stability and protein-protein interaction with cellular partners. Phosphorylation of proteins at serine, threonine and tyrosine residues is one of the most frequent forms of posttranslational modifications of proteins in eukaryotic cells. Many viral proteins are phosphorylated and their phosphorylation may play an important role in the viral infectious cycle. Some viral proteins are phosphorylated by virus-associated kinases. In some cases, phosphorylation of viral proteins by cellular kinase is a prerequisite for further phosphorylation by virus-associated kinases. Thus phosphorylation of viral proteins by cellular and/or viral kinases can be a critical step in viral pathogenesis.

Among cellular kinases, protein kinase CK2 (formerly casein kinase II) is one such kinases that has received special attention. CK2 is a pleiotropic, constitutive and ubiquitous serine/threonine protein kinase. Despite its constitutive nature, its upregulation or activation in the host cell has been linked with several viral pathogens in humans as well as plants. CK2 was first described as a tetrameric protein kinase that is composed of two catalytic subunits (CK2α and CK2α′) (38-42 kDa) and two β regulatory (CKβ) (27 kDa) subunits. CK2 is known to phosphorylate more than 300 proteins and is involved in signal transduction, transcriptional control, apoptosis, cell cycle regulation and cancer, and most recently in the regulation of cellular biological clock. HIV-1 Rev and Vpu, HSV-1 structural proteins VP22 and VP16, Epstein-Barr virus protein ZEBRA, KSHV protein ORF57, rabbis virus nucleoprotein, Herpes virus 6 E2, cytomegalovirus-early proteins IE2/IEP86 and Potivirus capsid are few of the notable substrates of CK2.

U.S. Patent Application Publication number 20040121968 published Jun. 24, 2004, which is incorporated herein by reference, discloses methods of inhibiting angiogenesis using a CK2 inhibitor.

### SUMMARY OF THE INVENTION

The present invention relates to methods of treating an individual infected with a virus selected from the group consisting of: West Nile Virus, Japanese encephalitis virus, Kunjin virus Tick-borne encephalitis virus and Hepatitis C virus. The methods comprise administering to such individual, a therapeutically effective amount of one or more compounds that inhibit CK2 activity, one or more compounds that inhibit CK2 expression or a combination thereof.

The present invention also relates to methods of treating an individual exposed to a virus selected from the group consisting of: West Nile Virus, Japanese encephalitis virus, Kunjin virus Tick-borne encephalitis virus and Hepatitis C virus. These methods also comprise administering to such individual, a therapeutically effective amount of one or more compounds that inhibit CK2 activity, one or more compounds that inhibit CK2 expression or a combination thereof.

The present invention further relates to pharmaceutical compositions comprising therapeutically effective amount of one or more compounds that inhibit CK2 activity, one or more compounds that inhibit CK2 expression, or a combination thereof.

The present invention also relates to methods of inhibiting viral replication by a virus selected from the group consisting of: West Nile Virus, Japanese encephalitis virus, Kunjin virus Tick-borne encephalitis virus and Hepatitis C virus. The methods comprise the step of contacting an antiviral composition selected from the group consisting of: one or more compounds that inhibits CK2 activity, one or more compounds that inhibits CK2 expression and combinations thereof, with cells that are infected with a virus selected from group consisting of: West Nile Virus, Japanese encephalitis virus, Kunjin virus Tick-borne encephalitis virus and Hepatitis C virus under conditions in which viral replication occurs in the absence of the antiviral composition.

The present invention further relates to methods of identifying a compound useful to treat infection by a virus selected from group consisting of: West Nile Virus, Japanese encephalitis virus, Kunjin virus Tick-borne encephalitis virus and Hepatitis C virus. The methods comprising the steps of performing a test assay in which a test compound is contacted with CK2 in the presence of a substrate, in conditions under which the CK2 phosphorylates the substrate in the absence of the test compound. The amount of phosphorylation observed in the test assay is compared with the amount of phosphorylation that occurs when CK2 is contacted with substrate in conditions under which the CK2 phosphorylates the substrate in the absence of the test compound. A lower amount of phosphorylation observed in the test assay compared to the amount of phosphorylation that occurs in the absence of the test compound indicates that the test compound inhibits CK2 activity. The test compound that inhibits CK2 activity is contacted with cells that are infected with a virus selected from group consisting of: West Nile Virus, Japanese encephalitis virus, Kunjin virus Tick-borne encephalitis virus and Hepatitis C virus under conditions in which viral replication occurs in the absence of the test compound. The level of viral replication that occurs to the presence of the test compound is compared with the level of viral replication that occurs to the absence of the test compound. A reduction of the level of viral replication that occurs to the presence of the test compound compared to the level of viral replication that occurs to the presence of the test compound indicates that the compound is useful to treat infection by a virus selected from group consisting of: West Nile Virus, Japanese encephalitis virus, Kunjin virus Tick-borne encephalitis virus and Hepatitis C virus.

The present invention also relates to methods of identifying a compound that inhibits CK2. The methods comprise the steps of performing a test assay in which a test compound is contacted with CK2 in the presence of a substrate, in conditions under which said CK2 phosphorylates the substrate in the absence of said test compound. The amount of phosphorylation observed in the test assay with is compared to the amount of phosphorylation that occurs when CK2 is contacted with substrate, in conditions under which CK2 phosphorylates the substrate in the absence of the test compound. The substrate is selected from the group consisting of: West Nile Virus C protein, Japanese encephalitis virus C protein, Kunjin virus C protein, Tick-borne encephalitis virus C protein and hepatitis C virus NS2/NS3 protein. A lower amount of phosphorylation observed in the test assay compared to the amount of phosphorylation that occurs in the absence of the test compound indicates that the test compound inhibits CK2 activity.

### BRIEF DESCRIPTION OF THE FIGURES

FIG. 1 includes panels A, B and C. FIG. 1 panel (A): Purification of His-tagged rC from WNV. The left panel indicates the coomaassie staining of the gel depicting different steps of protein purification. The right panel indicates the western blotting of the gel using monoclonal anti-His antibody. Appearance of two bands from the Ni-TA-fraction suggests that capsid protein may predominantly exist as a dimmer (lanes 6). Cross-reaction of unknown sample from the crude preparation to anti-His antibody is marked by *. The position of the protein markers is indicated on the left side. FIG. 1 panel (B) WNV C is phosphorylated by CK2 in vitro. M15 E. coli lysate containing expressed C as well as the purified C were subjected to CK2 assay. They are phosphorylated only when radio-labeled phosphosource and rCK2 are provided. The samples were not phosphorylated in the absence of recombinant CK2 and hence C is not an auto-phosphorylable protein. FIG. 1 panel (C) The protein C accepts phosphatidyl moiety from both ATP and GTP P32 labeled ATP as well as GTP were provided as phosphate donors in the kinase assay. Also the assay was carried out under different concentrations of Heparin, which is a CK2 inhibitor.

FIG. 2 includes panels A-D. FIG. 1 panel (A): Neither CK2α nor CK2β alone can phosphorylate the protein C. Purified recombinant CK2α and CK2β subunits were evaluated for their ability to phosphorylate the protein C. The first lane indicates the phosphorylation by CK2 whole enzyme. Under similar settings, purified recombinant protein of CK2α or CK2β failed to phosphorylate the capsid protein (lanes 2 and 3). FIG. 1 panel (B): Inhibition of in vivo phosphorylation of C by DRB. The cells were transfected with pcWNV-C-His plasmids and pulse chased with [32P]phosphoric acid under the treatment with different concentrations of DRB. The lysates were immunoprecipitated with anti-H antibody and the precipitated samples were analyzed by autoradiography as described in Materials and Methods. Capsid-transfected matching samples were maintained with [³²P]phosphoric acid exposures. The lysates from these samples were analyzed in western blotting with anti-actin to validate the loading controls (right panel). FIG. 1 panels (C) and (D): CK2α and CK2β-siRNA inhibit in vivo phosphorylation (C and D). The cells were transfected with siRNA molecules that specifically inhibit the expression of endogenous CK2α and CK2β. The cells were harvested and their lysates were subjected to western blotting analysis by anti-CK2α and anti-CKβ antibodies individually. In comparison with the untransfected control (Lane 1; C), CK2α-siRNA-transfected cells exhibited a reduced level of expression of CK2α (Lane 2; C). Considering the level of CKβ from the untransfected samples (lane 3; C), addition of CKβ-siRNA to the cells completely inhibited the expression of CKβ (lane 4; C). FIG. 1 panel (D): CK2-siRNAs inhibit in vivo C phosphorylation. pcWNV-C-His-transfected cells were metabolically labeled with [³²P]phosphoric acid and the level of in vivo phosphorylation was assessed with or without the influence of CK2α-siRNA or CKβ-siRNA. The addition of Both siRNA-CK2 molecules completely inhibited the in vivo phosphorylation of C (lanes 3 and 6; D) Immunoprecipitation of ³²P-labeled C from untreated matching controls and lack of signal from the mock-treated samples demonstrates the specificity of this assay.

FIG. 3 includes panels A, B and C. The CK2 consensus motif (SLID) is shown life panel in FIG. 3 panel A. Alanine substitution is shown at the corresponding Serine residue is boxed. The mutant capsid protein, C(S36A) is critically impaired in its ability to get phosphorylated by CK2 kinase. FIG. 3, panel B indicates the sequence of peptides derived from C. Three different regions were chosen. The CK2 consensus motif is boxed on the peptide sequence of Cp(30-48). The CKII-Pep is substrate peptide obtained from the commercial source. These peptides were assayed for CK2-assay and analyzed by dot-blot analysis. Uniform amount of peptide samples were loaded onto the membrane as described in Material and methods. The blot was assayed for CK2 assay. Only Cp(30-48) and CK-II-Pep peptides reacted positively to CK2 assay (FIG. 3 panel C; lanes 2 and 4).

FIG. 4 includes Panels A-D. WNV C interacts in vitro and in vivo with CK2α. CK2α and CK2β cDNA were amplified from HeLa cell cDNA and cloned into pcDNA3.1. pcCK2α and pcCKβ plasmids encoding CK2α and CK2β subunits respectively were used in the binding assay as described in the Materials and Methods. The lanes 1 and 2 indicates the inputs ³⁵S-labeled CK2α and C proteins respectively. Both of these protein samples were co-immunoprecipitated by addition of either anti-His (targeting His-tag of C) or anti-CK2α (lanes 3 and 4). The lanes 5 and 6 reveal the sample inputs for CK2β and the C samples respectively. Anti-His antibody was able to precipitate only C and not the CK2α from the mix containing both CK2β and C; also, anti-CK2β was able to precipitate only CK2β and not the C. Thus in vitro, capsid is interacted only by CK2α and not by CK2β. Interaction between the C and CK2 in vivo: 293-T cells were transfected with C and/or CK2α/CK2β expression plasmids and analyzed by Immunoprecipitation followed by Western blot analysis as described in Materials and methods. The lane 1 indicates the co-precipitation of CK2α by C immunoprecipitated by anti-His antibody. Though pcCK2α was not added in this lane, C was sufficient to precipitate the endogenous level of CK2α. Similar trend was noted from the cells that express both WNV C and CK2α. When anti-CK2α antibody was used to immunoprecipitate, both C and CK2α, were detected. The bottom panel of western blotting indicates the input controls. Results are representative of two independent experiments.

FIG. 5 includes panels A and B. FIG. 5 panel A: C(S36A) is less efficiently incorporated into VLPs than C(wt). The ability of C (wt) and C(S36A) capsid proteins to participate in VLP production was evaluated. As described in materials and methods, the cells were transfected with a cocktail of plasmids encoding and E and preM and C (wt) or C(S36A) or empty vector. Forty-eight hours post-transfection, the supernatants were collected and analyzed for viral budding. FIG. 5 panel A shows the ELISA data using anti-C antibody, indicating absorbance values at 450 nm and reflect O.D. for each sample minus the background wells (carbonate bicarbonate buffer). The data reveals the presence of higher amount of capsid antigens only from the culture fluid of cells transfected with combination of plasmids that includes C(wt) (lane 3) than that of the samples containing C9S36A) (lane 2). The capsid was not detected from the negative controls that included vector alone instead of any capsid construct (1). FIG. 5 panel B: Mutant capsid results in Reduced level of capsid-containing Viral-like-particles (VLP) release. A portion of the transfected cells from similar the experimental setting were subjected to EM analysis. Representative cells co-transfected with the mixture of plasmids that included empty vector (a), C(Wt) (b-c), and C(S36A) (d) are shown here. The panels b and c shows the accumulation and release of VLPs assembled beneath the outer membrane area. These figures also explicitly show these VLPs budding from the outer membrane of the cells and released into the extracellular medium. The production of VLP was significantly removed (d) from the cells that included C(S36A) construct.

FIG. 6 includes panels A and B. Both reduced CK2 activity and depletion of CK2 transcripts affects viral release. FIG. 6 panel A: Depletion of CK2 transcripts reduced viral replication. As described earlier, the cells were transfected with full-length infectious clone along with siRNA-CK2α and siRNA-CK2β molecules. Lane 1 depicts the amount of viral release from the plates that were not treated with any of siRNA molecules. In the lane 2 and 3, only the samples that included siRNA-CK2 molecules exhibited a reduced level of viral release by about one fold (solid bars) in comparison with the samples that received negative control siRNA-molecules (empty bars). FIG. 6 panel B: Effect of cell-permeable CK2-inhibitor on viral release. 293 cells were infected with infectious WNV samples, propagated from an infectious clone of WNV. The infected cells were maintained with or without different doses of DRB in the medium and forty-eight hours after infection, the culture fluids were analyzed for the presence of WN viral antigen, E. Addition of DRB to the medium even at the concentration of 5 uM significantly reduces viral release and its effect is almost dose dependent.

FIG. 7 includes panels A and B. Plaque assay of WNV titers from DRB and siRNA-CK2 treated WNV infected cells. These data measures the level of viral release from the samples as described in FIG. 6, by plaque assay. FIG. 7 panel A reveals that the depletion of mRNA transcripts of CK2α and CK2β by siRNA transcripts reduces the amount of viral release by almost one fold. The cells that were treated with negative control siRNA molecules, exhibited an amount of viral release resembling level of untreated controls. The inhibited level of viral release indicates the siRNA-CK2 mediated effects are statistically significant. The data in FIG. 7 panel B reveals the effect of the addition of DRB to the medium on WNV replication and subsequent release into culture medium. In consistent with the ELISA data, the effect of DRB appears to be dose dependent as clearly revealed here (Lanes 2-5). Except the effect of DRB at 5 uM, the rest of the values are statically significant as determined by p-values.

### DESCRIPTION OF PREFERRED EMBODIMENTS

WNV C is phosphorylated in vitro as well as in vivo by CK2. CK2-specific inhibitors inhibit the phosphorylation of this viral antigen. Under the influence of specific siRNA molecules targeting CK2α and CK2β, in vivo phosphorylation of C is completely inhibited. Furthermore, the phosphorylation site in WNV C was mapped to Serine 36 as confirmed by site-directed mutagenesis and dot-blot kinase assay involving C-derived peptides. Most importantly the first functional consequence of constitutive phosphorylation of C has been elucidated: phosphorylation of Ser-36 has a critical role in the ability of the protein C to participate in virion formation.

In addition to WNV, other flaviviral members have C proteins with regions that correspond to that of the WNV C including the phosphorylation site whose status is critical to virion formation. The phosphorylation of the C protein from WNV, as well as phosphorylation of the C protein from any other flaviviral members with a corresponding phosphorylation site such as Japanese encephalitis virus and Kunjin virus (Australian WNV subtype), is critical to virion formation and can be inhibited by CK2 inhibitors or by inhibiting expression of functional CK2 in order to treat infections by these viral pathogens.

According to one aspect of the invention, C protein phosphorylation can be inhibited by CK2 inhibitors and this inhibition results in an inhibition of virion formation. Thus, the present invention provides compositions comprising CK2 inhibitors and methods of using CK2 inhibitors to treat individuals infected with WNV, JEV or Kunjin virus and methods of preventing viral infections in individuals exposed to or suspected of being exposed to WNV, JEV or Kunjin virus. “Preventing of viral infection” is meant to refer to the inhibition or reduction in the establishment of a viral infection in an individual such that the individual does not develop symptoms. “Treatment of viral infection” is meant to refer to the reduction or amelioration of symptoms and level of viral replication in individuals who have been infected with WNV, JEV or Kunjin virus.

According to another aspect of the invention, C protein phosphorylation can be inhibited by inhibiting expression of functional CK2. This inhibition results in an inhibition of virion formation. Thus, the present invention provides compositions comprising compounds which inhibit CK2 expression and methods of using CK2 expression inhibitors to treat individuals infected with WNV, JEV or Kunjin virus and methods of preventing viral infections in individuals exposed to or suspected of being exposed to WNV, JEV or Kunjin virus. CK2 expression can be inhibited by inhibiting expression of either or both of CK2α and CKβ. Expression can be inhibited using antisense or siRNA technology targeted at one or more of CK2α, CK2α′ and CKβ.

Amino acid sequences of Kunjin virus C proteins are shown in SEQ ID NO:1 and SEQ ID NO:2 (Genbank accession number AAP78942 and AAP78942, which are incorporated herein by reference).

Amino acid sequences of JEV C proteins are shown in SEQ ID NO:3 and SEQ ID NO:4 (Genbank accession number AAC02714 and AAC29474, which are incorporated herein by reference).

The amino acid sequences of Tick-borne encephalitis virus C proteins is shown in SEQ ID NO:5 (Genbank accession number AAF8224, which is incorporated herein by reference).

In each case, the protein sequence contains a sequence homologous to the WNV C protein phosphorylation site which includes Serine 36 (Amino acid Serine 36 in each of SEQ ID NOs:1-4 and amino acid Serine 36 in SEQ ID NO:5.

CK2 inhibitors that selective inhibit CK2 such that a reduction of CK2 enzymatic activity occurs in its presence compared to an appropriate control that lacks the CK2 inhibitor. CK2 inhibitors include a diverse group of commercially available compounds. Examples of such compounds include: DRB, 5,6-Dichloro-1-b-D-ribofuranosylbenzimidazole, 5,6-Dichlorobenzimidazole riboside, (BIOMOL Research Laboratories, Plymouth Meeting, Pa., USA, catalog #E1231); TBB, TBBt, 4,5,6,7-Tetrabromo-2-azabenzimidazole, 4,5,6,7-Tetrabromobenzotriazole (Calbiochem, San Diego, Calif., USA, Catalog #218697, also available as Calbiochem InSolution™ Catalog #218708); DMAT, 2-Dimethylamino-4,5,6,7-tetrabromo-1H-benzimidazole (Calbiochem, San Diego, Calif., USA, Catalog #218699, also available as Calbiochem InSolution™ Catalog #218706); M6760 Myricetin, 3,3′,4′,5,5′,7-Hexahydroxyflavone Cannabiscetin, CAS #529-44-2, Beilstein Registry #332331, MDL #MFCD00006827 (SIGMA-ALDRICH); A-3.hydrochloride, n-(2-Aminoethyl)-5-chloronapthalene-1-sulfonamide.HCl (Axxora Life Sciences, San Diego, Calif., USA Catalog #ALX-270-039); emodin, 3-methyl-1,6,8-trihydroxyanthraquinone, 6-methyl-1,3,8-trihydroxyanthraquinone, Beilstein Registry #1888141) and aloe-emodin, 1,8-dihydroxy-3-hydroxymethylanthraquinone.

Antisense and siRNA techniques are well known and one skilled in the art can readily design antisense oligonucleotides or siRNA oligonucleotides which can inhibit expression of CK2α, CK2α′ or CK2β. mRNA sequences for CK2α are Genbank accession number NM177559, NM001895 and NM177560, which are each incorporated herein by reference. The mRNA sequence for CK2α′ is Genbank accession number NM001896, which is incorporated herein by reference. The mRNA sequence for CK2 β is Genbank accession number NM001320, which are each incorporated herein by reference.

One skilled in the art can diagnose an individual infected with WNV, JEV or Kunjin virus. Upon making such diagnosis, the individual can be administered an amount of one or more CK2 inhibitors and/or one or more CK2 expression inhibitors to inhibit C protein phosphorylation thereby inhibit virion formation and treating the individual. Individuals who are known to have been exposed to WNV, JEV or Kunjin virus, or who are likely to have been in contact with the virus by virtue of contact with an infected animal, person or contaminated item, as well as individuals who are likely to be exposed to WNV, JEV or Kunjin virus, or who are likely to come into contact with the virus by virtue of expecting to come into contact with an infected animal, person or contaminated item, can be administered an amount of one or more CK2 inhibitors and/or one or more CK2 expression inhibitors to inhibit C protein phosphorylation thereby inhibit virion formation and prevent infection of the individual.

The methods of the invention may be used in combination with other antiviral methods.

Pharmaceutical compositions may be formulated by one having ordinary skill in the art with compositions selected depending upon the chosen mode of administration. Such compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985) a standard reference text in this field, which is incorporated herein by reference. Pharmaceutically acceptable carriers are those that are compatible with the other ingredients in the formulation and biologically acceptable.

The pharmaceutical compositions of the present invention may be administered by any means that enables the active agent to reach the agent's site of action in the body of a mammal. Pharmaceutical compositions may be administered orally or parenterally, i.e., intravenous, subcutaneous, intramuscular, etc. The compounds of this invention may be administered neat or in combination with conventional pharmaceutical carriers, the proportion of which is determined by the solubility and chemical nature of the compound, chosen route of administration and standard pharmacological practice. The pharmaceutical carrier may be solid or liquid.

Applicable solid carriers can include one or more substances which may also act as flavoring agents, lubricants, solubilizers, suspending agents, fillers, glidants, compression aids, binders or tablet-disintegrating agents or an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions which are sterile solutions or suspensions can be administered by, for example, intramuscular, intraperitoneal or subcutaneous injection. Sterile solutions can also be administered intravenously. Oral administration may be either liquid or solid composition form.

The compounds of this invention may be administered rectally or vaginally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may also be administered transdermally through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired. The dosage requirements vary with the particular compositions employed, the route of administration, the severity of the symptoms presented and the particular subject being treated. Based on the results obtained in the standard pharmacological test procedures, projected daily dosages of active compound would be 0.02 μg/kg-750 μg/kg. Treatment will generally be initiated with small dosages less than the optimum dose of the compound. Thereafter the dosage is increased until the optimum effect under the circumstances is reached; precise dosages for oral, parenteral, nasal, or intrabronchial administration will be determined by the administering physician based on experience with the individual subject treated.

According to some aspects of the invention, methods are provided for identifying compounds useful to treat WNV, JEV and Kunjin virus. The methods comprise the steps of identifying a compound that inhibits CK2 activity and testing such CK2 inhibitors to determine their effect on viral replication of WNV, JEV and Kunjin virus.

In some embodiments the methods comprise contacting CK2 enzyme and a CK2 substrate in the presence of a test compound under conditions in which the CK2 substrate would be phosphorylated in the absence of a test compound. If the phosphorylation of the CK2 substrate is reduced in the presence of the test compound as compared to the level of phosphorylation in the absence of the test compound, the test compound is a CK2 inhibitor. According to some embodiments of the invention, the cells permissive for infection by WNV, JEV or Kunjin virus and capable of supporting viral replication are contacted with WNV, JEV or Kunjin virus in the presence of the CK2 inhibitor under conditions in which the cells would become infected and viral replication would occur in the absence of the CK2 inhibitor. If the level of viral replication is reduced in the presence of the CK2 inhibitor compared to the level of viral replication that occurs in the absence of the CK2 inhibitor, the CK2 inhibitor is useful as an antiviral agent for the treatment WNV, JEV or Kunjin virus exposure and infection according to the invention.

The CK2 substrate may be any substrate known to be phosphorylated by CK2. In some embodiments, the CK2 substrate is a C protein from WNV, JEV or Kunjin virus.

In some embodiments the methods comprise contacting more than one test compounds, in parallel. In some embodiments, the methods comprises contacting 2, 3, 4, 5, 6, 7, 8, 9, 10, 15, 20, 25, 30, 40, 50, 100, 1000, at least 2, at least 5, at least 10, at least 50, at least 100, or at least 1000 test compounds in parallel. In some embodiments, the present invention uses High Throughput Screening of compounds and complete combinatorial libraries can be assayed, e.g., up to thousands of compounds. Methods of how to perform high throughput screenings are well known in the art. The methods can also be automated such that a robot can perform the experiments. The present invention can be adapted for the screening of large numbers of compounds, such as combinatorial libraries of compounds. Indeed, compositions and methods allowing efficient and simple screening of several compounds in short periods of time are provided. The instant methods can be partially or completely automated, thereby allowing efficient and simultaneous screening of large sets of compounds.

Positive and negative controls may be performed in which known amounts of known CK2 inhibitors and no compound, respectively, are added to the assay. One skilled in the art would have the necessary knowledge to perform the appropriate controls.

The test compound can be any product in isolated form or in mixture with any other material (e.g., any other product(s)). The compound may be defined in terms of structure and/or composition, or it may be undefined. For instance, the compound may be an isolated and structurally-defined product, an isolated product of unknown structure, a mixture of several known and characterized products or an undefined composition comprising one or several products. Examples of such undefined compositions include for instance tissue samples, biological fluids, cell supernatants, vegetal preparations, etc. The test compound may be any organic or inorganic product, including a polypeptide (or a protein or peptide), a nucleic acid, a lipid, a polysaccharide, a chemical product, or any mixture or derivatives thereof. The compounds may be of natural origin or synthetic origin, including libraries of compounds.

The amount (or concentration) of test compound can be adjusted by the user, depending on the type of compound (its toxicity, cell penetration capacity, etc.), the number of cells, the length of incubation period, etc. In some embodiments, the compound can be contacted in the presence of an agent that facilitates penetration or contact with the cells. The test compound is provided, in some embodiments, in solution. Serial dilutions of test compounds may be used in a series of assays. In some embodiments, test compound(s) may be added at concentrations from 0.01 μM to 1M. In some embodiments, a range of final concentrations of a test compound is from 10 μM to 100 μM.

### Example

### Materials and Methods

Cells, viruses and viral infections. African green monkey kidney cells (Vero) and 293T cell lines were obtained from ATCC (Manassas, Va.). Culture media and other standard tissue culture reagents were obtained from Life Technologies, Inc., (Rockville, Md.). Vero cells were grown in minimal essential medium supplemented with 10% fetal bovine serum, 2 mM L-glutamine, 1% sodium pyruvate, and 1% penicillin/streptomycin.

Reagents and Plasmids. The mammalian expression vector, pcDNA3.1/His A, Top 10F E. coli chemically treated competent cells and anti-His monoclonal antibody were purchased from Invitrogen, (Carlsbad, Calif.). In vivo labeling-grade ³⁵S-methionine was purchased from Amarsham Pharamacia Biotech. Monoclonal anti-actin antibody was purchased from Sigma Co(St. Louis, Mo.). Histone H1 and purified CK2β subunit proteins were from Calbiochem-Novabiochem Corporation (La Jolla Calif.). Anti-CK2α subunit, recombinant CK2, CK2α subunit proteins were received from Biomol Research Laboratories (Plymouth Meeting, Pa.).

Construction of plasmids encoding WNV capsid and human CK2 subunits. The chimeric WNV capsid with a C-terminal polyhistidine tag was cloned from a WNV capsid-vaccine construct that has been described earlier. Using this construct as a template, the capsid open reading frame was amplified and the eluted fragment was cloned into pcDNA3.1N5/HisA expression vector. The forward and reverse primers for cloning capsid were 5′-ACCATGTCTAAGAAACCAGGAGGC-3′ and 5′-TGCTCCTA-CGCTGGCGATCAGGCC-3′ respectively. The capsid mutants were constructed by overlapping PCR protocol with appropriate internal primers using standard procedures. CK2α and CK2β encoding cDNAs were prepared from total cDNA prepared from HeLa cells. The forward and reverse primers for amplifying CK2α ORF were ATGTCGGGACCCGTGCCAAGC and TTACTGCT-GAGCGCCAGCGGCAGC respectively; the forward and reverse primers required for the amplification of CK2β fragment were ATGAGCAGCTCAGAGGAGGTGTCC and TCAGCGAATCGTCTTGACTGGGCTCTT. The amplified fragments of CK2α and CK2β were cloned into pcDNA3.1 vector and the resulting plasmids are referred as pCK2α and pCK2β respectively. The integrity of the constructs was confirmed by automated sequencing.

Recombinant protein expression. Capsid (His)6 proteins were expressed in Escherichia coli strain M15[pREP4] cells and purified using nickel-nitrilotriacetic acid (Ni2+-NTA) agarose (Qiagen, Valencia, Calif.). The cDNA of the wild-type WNV Cp and its mutants were cloned into the Bam Hi site of the bacterial expression vector pQE30. Resulting constructs pQE30-WNV-Cp(Wt) and pQE30-WNV-Cp(S36A) were used to transform E. Coli strain M15[pREP4] cells. The transformants were screened onto LB-Amp-Kan plates. Single positive clone was identified and confirmed for the presence of Cp. Transformants were grown in LB-Amp-Kan media overnight, induced with isopropylthio-B-galactoside for 2-4 hours and harvested by centrifugation. The suspension of cells were suspended in buffer R1 (100 mM NaCl and 50 mM Tris-HCl, pH 8.0). The suspension was passed three times at 90 MPa through a prechilled French pressure cell. Purification of (His)₆ Cp was performed using chromatography on (Ni²⁺-NTA) agarose. The lysate was centrifuged at 40,000 g for 45 min, and the supernatant fraction was processed by Ni²⁺-NTA agarose chromatography under native conditions. Proteins were eluted with a linear 0.1 to 1 M NaCl gradient buffered with 50 mM Tris-HCl, pH 8.0. Purified (His) 6tCK2 was stored at +4° C. for up to 1 month without detectable loss of activity or protein integrity. Aliquots of protein samples were analyzed by SDS-PAGE to determine the level of purity. The purified samples were used for further Kinase assays.

SDS-PAGE and western blot analysis. Protein were analyzed by SDS polyacrylamide gel electrophoresis according to the procedure of Laemmli (1971). After drying the gels, phosphorylation of proteins was visualized by autoradiography. For Western Blot, proteins were transferred to a PVDF membrane by tank blotting with 20 mM Tris/Hcl (ph 8.7) and 150 mM glycine as transfer buffer. Membranes were blocked in phosphate buffered saline (PBS) with 0.1% Tween-20 and 5% dry milk for 1 h at room temperature. The membrane was incubated with the primary antibody in PBS Tween-20 with 1% dry milk for 1 h. The membrane was then washed with PBS-Tween-20 three times before incubating with the peroxidase—coupled secondary antibody in a dilution of 1:30,000 in PBS—Tween-20 with 1% dry milk. Signals were developed and visualized by the chemiluminescent system (Amersham Pharmacia Biotech).

In vitro protein kinase assays. In vitro phosphorylation assays were routinely done with the recombinant Cp proteins with the recombinant CK2 (Biomol, Pa.) in a 20 μl-reaction volume. Reaction mixtures (20 μL) contained 25 mM Hepes, pH 7.4, 2 mM MnCl₂ or 5 mM MgCl₂, 1.6 μCi of -³²P-GTP or -33 P-ATP (Amersham Pharmacia Biotech), and 0.5 to 1 μg of purified His-tagged WNV Cp as a substrate. Histone 1B (Calbiochem) protein was used as positive control. The phosphorylation reaction was started by the addition of ³²P-ATP or ³²P-GTP and the reaction was carried out for 30 min at 30 C. The kinase assays were arrested by the addition of 20 μl of SDS-sample buffer and the reaction mixtures were boiled for five minutes. Phosphorylated proteins were separated in SDS PAGE and proteins ere visualized by autoradiography.

Dot-blot kinase assay using WNV C peptides. The C peptides representing three different domains were synthesized and HPLC-purified. The peptides were dissolved in DMSO and immobilized onto Pro Blott membranes (Applied Biosystems) using Dot Blot system. The membrane was equilibrated overnight in kinase buffer supplemented with 1% bovine serum albumin (BSA). For the phosphorylation of the peptides, the membrane was incubated in 2 ml kinase buffer with recombinant CK2 in the presence of ³²P-ATP. After 30 min at 37 C, the membrane was washed three times with 1M NaCl and subsequently with urea containing buffer to remove any bound protein. Finally the membrane was washed with ethanol and dried. Phosphorylated peptides were visualized by autoradiography. Upon capturing the autoradiographic image, the blots were stained with Coomaassie blue-R250 and photographed.

siRNA protocols. SiRNA molecules prepared against CK2α and CK2β subunits and siRNA transfection reagents and siRNA transfection media were purchased from Santa Cruz Biotechnology (San Diego, Calif.). Transfections with siRNA reagents were carried out as per the protocols provided by the suppliers. Typically the cells were grown in 6-well plates and grown overnight to achieve 60% confluency. Five microliter of 10 μM siRNA was added to 75 μl of siRNA transfection medium and the contents were gently mixed and kept at room temperature for 5 min. Separately, 5 μl of SiRNA transfection reagent was added to the 20 μl of siRNA transfection medium and the contents were mixed and set aside for 5 min. Both siRNA and siRNA-transfection medium mixtures were mixed together and incubated at room temperature for 20 min. The media were removed from cells and 1500 μl of fresh medium were added to each well. After sufficient incubation, siRNA-transfection reagent complex were gently added dropwise to well while gently rocking the plates. Transfected cells were incubated in appropriate conditions.

In vivo ³²P-Orthophosphate labeling and WNV C immunoprecipitation. In order to check whether Cp is phosphorylated in vivo also, Capsid-transfected cells were pulse-chased with ³²P-ATP and their lysates were analyzed for the detection of phosphorylated capsid proteins by Immunoprecipitation. HeLa cells were grown in DMEM medium supplemented with 10% FBS until they were subconfluent. The cells were transfected with WNV Cp constructs and DOTAP/DNA containing media were replaced after about 6-hours with media with/without DBS. DBS was added to a final concentration of 0.1, 0.5 and 1.0 mM. The Control cells were treated with DMSO alone. Phospholabeling and immunoprecipitation of WNV CP were performed as described previously. To assess the in vivo effect of the CK2 inhibitor 5,6-dichloro-14-D-ribofuranosyl)benzimidazole (DRB; Calbiochem) on WNV CP phosphorylation, the compound was added at a final concentrations of 100, 500 and 1000 μM. The stock solution of DRB was prepared in DMSO; therefore, DMSO was present in the phospholabeling solution at a final concentration of 0.5% (v/v). WNV CP was precipitated with anti-His monoclonal antibody (Scil Diagnostics, Martinsried, Germany). NET buffer (50 mM Tris-HCl, pH 8.0, 150 mM NaCl, 50 mM NaF, 5 mM EDTA, pH 8.0, 0.5% [v/v] Nonidet P-40, and 0.02% sodium azide) was supplemented with 2% BSA to reduce nonspecific antibody binding. Protein G—Sepharose (Amersham Pharmacia Biotech) was used as an immunoadsorbent. Antibodies were first prebound to protein G-Sepharose and then incubated with the cell lysate containing WNV CP. This modification allowed the removal of unbound antibodies from the incubation mixture before the addition of the antigen. The immunoprecipitated capsid proteins were eluted from the protein G-agarose by boiling them for 5 min and analyzed by SDS-PAGE, followed by autoradiography.

In vitro ³⁵S-labeling and autoradiography. In order to carry out in vitro binding assays, ³⁵S-labeled protein samples of CK2 subunits and capsid were generated from their expression plasmids. TNT coupled in vitro transcription/translation system (Promega Corporation, Madison, Wis.) was employed as per instructions supplied by the manufacturer. An aliquot of in vitro translated capsid and CK2 proteins were m incubated in the binding buffer on ice for 90 min. Further either anti-His or anti CKII-antibodies were mixed with the protein samples and the resulting mix incubated further for 90 min at 4° C. Approximately five-milligram of Protein A-Sepharose (Amarsham Pharmacia Biotech) beads was added to this mix from a freshly prepared stock (100 mg/ml) and the samples were further incubated at 4° C. for 90 min in a rotating shaker. The beads were then washed once with binding buffer and twice with RIPA buffer and finally suspended in 2×SDS sample buffer. The immunoprecipitated protein samples were eluted from Sepharose beads by briefly boiling them and the samples were resolved by SDS/PAGE. The gel was fixed, treated with a 1M sodium salicylate solution to enhance the signal and dried in a gel drier (Biorad, Hercules, Calif.). The dried gel was exposed overnight to X-ray film and developed using an automated developer (Kodak, Rochester, N.Y.).

Quantification of the protein C in ELISA from the supernatants of transfected cells. The ability of unprocessed and truncated capsid proteins to participate in the budding was assed by ELISA from culture fluid of transfected cells. In 6 cm plates, the cells were transfected with full-length capsid (123 aa) or truncated capsid (105 aa) or C(S36A) plasmids or in combination with WNV M and E-expressing constructs. Forty eight hours post-transfection, the supernatants were collected and analyzed for viral budding. One hundred microliters of supernatants were plated in triplicate, in a serial dilution series (dilutions in carbonate bicarbonate buffer) on a high protein binding EIA/RIA 96 well plate (Costar) and incubated at 4° C. overnight. In addition to the supernatants from experimental samples, a mock-transfected and untransfected media control is also added to the assay samples. Moreover, a series of wells are used as negative controls for the assay in that no supernatant is added to these wells only carbonate bicarbonate buffer. After 3 washes with PBS, nonspecific binding sites are blocked with 3% BSA in PBS solution, for 2 hours at room temperature. After washing with PBS, 100 microliters of monoclonal anti-C antibody is added to the wells at a 1:500 dilution in PBS/1% BSA diluent and incubated for 1 hour at 37° C. The excess primary antibody is removed by washing with PBS and 100 microliters of anti-mouse secondary-horse radish peroxidase (HRP) labeled is added to each well at a 1:100 dilution in PBS/1% BSA diluent. TMB is used as a substrate to detect HRP and is incubated for 30 minutes. The reaction is terminated by adding 4N H₂SO₄ and protein levels are measured using an ELISA reader (Molecular Diagnostics) at 450 nm. The data were shown as O.D. values at 450 nm and reflect O.D. for each sample minus the background wells (carbonate bicarbonate buffer).

Virus titration by plaque assay. Virus production was titered by plaque assays using Vero cells. For routine analysis, cells were seeded in 6-well (6×10⁵ cells/well) or 12-well (3×10⁵ cells/well) plates in -MEM with 10% FBS for 3 h at 37° C. Medium was removed, serial dilutions of virus supernatants in -MEM with 2% FBS were added (0.30 ml/well for 6-well plates and 0.15 ml/well for 12-well plates), and the cells were incubated for 2 h at 37° C. Subsequently, -MEM containing 5% FBS and 1% low-melting-point agarose (SeaPlaque; FMC Bioproducts, Rockland, Me.; 3 ml/well for 6-well plates and 1.5 ml/well for 12-well plates) was added, and the plates were incubated at 37° C. for 5 days. The plaques are visualized after 10% formaldehyde fixation (>1 h at room temperature) and removal of the agarose plug by staining briefly (15 to 30 seconds) with a 1% crystal violet solution in 20% ethanol. Virus concentrations are determined as PFU per milliliter.

Statistical analysis. All values are expressed as mean±standard error of the mean. A student t test was used to determine the statistical significance, with values of P<0.05 considered significant.

Electron microscopic (EM) analysis of transfected cells. Vero cells, grown in 6-cm plates, were transfected with sets of plasmids encoding C-His or C(S36A) and preM and E. Untransfected and pcDNA3.1 vector transfected cells were maintained parallely as negative controls. Forty either hours post transfection, the cells were harvested and washed twice with PBS, and fixed with 3% glutaraldehyde in 0.300 mM sodium phosphate buffer, pH.7.4. The samples were treated with 1% osmium tetroxide, dehydrated with acetone and embedded in Epon 612 resin. The specimens were examined in a transmission electron microscopy at 80 kV. The images were acquired using ImagePro 6.7 software and further processed in Adobe Photoshop 7.0 software.

### Results

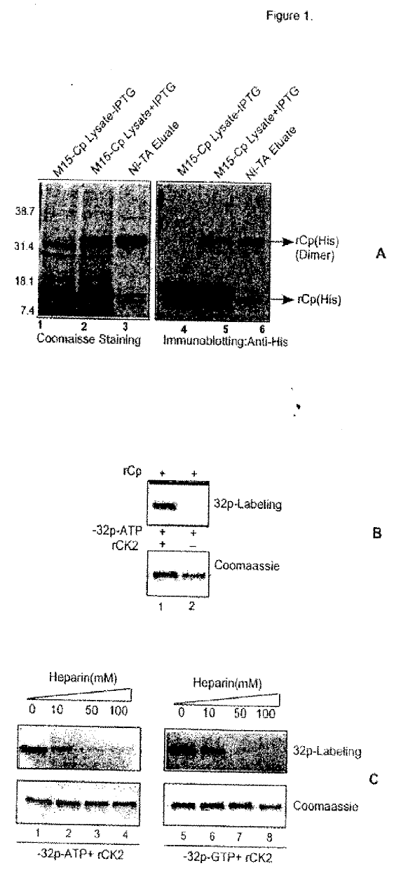

Expression and purification of recombinant WNV C. Recombinant capsid protein from WNV was expressed in bacteria and purified for analysis in kinase assays. For this purpose, the open reading frame encoding WNV C was cloned in frame with a region coding for an hexa-histidine affinity tag added at the amino terminus using the pQE-30 expression vector (FIG. 1A). The E. coli strain M15[pREP4] harboring chimeric capsid construct produced a detectable amount of rC. From the lysates of M15[pREP4+pQE30-C], rC was purified using nickel-nitrilotriacetic acid (Ni²⁺-NTA) agarose (Qiagen, Vencia, Calif.). The Ni-NTA eluates were passed through Centicon dialysis column (Millipore). In FIG. 1A, The gel analysis of Ni-NTA-eluates column indicated a denser band, compared to the positions of standard proteins, that corresponds to the mass of about 33 kDa and a lighter bands at the position of 14 kDa (lane 3, C). The same fractions were also analyzed by western blotting using monoclonal anti-His antibody that detects specifically fusion proteins that bear N-terminal hexa-histidine tag. The right-panel in FIG. 1C reveals the western analysis of the eluates containing recombinant WNV C. Two prominent signals corresponding to the positions at 14 kD and 33 kD are visible from the eluates of Ni-NTA column indicating the elusion of the capsid protein. The positions of these western signals match the coomassie staining patterns of NI-NTA elutes as appeared in lane 3. Besides the signals specific for capsid, we observed a stronger non-specific signal above the position of capsid protein from both uninduced and induced crude E. coli lysates (marked with *). However these non-specific signals were absent from the purified fractions.

WNV C is phosphorylated by CK2 in vitro. Purified rC fractions from E. coli cells (M15[pREP4+pQE-WNV C]) were subjected to subsequent kinase assays. Using purified CK2 enzyme, in vitro assay was performed with ³²P-ATP as a phosphate donor to examine possible role of CK2 in the phosphorylation of WNV C. FIG. 1B reveals that purified fractions yielded was phosphorylated by CK2. Also, protein C was not phosphorylated when CK2 kinase was not added to the kinase reaction mix. CK2 is unique among protein kinases in its ability to use both ATP and GTP as phosphoryl group donors. Another biochemical property of CK2 that makes it different from other kinases is its high sensitivity to inhibition by heparin. Hence, the phosphorylation of C was studied in the presence of ³²P-labeled both GTP (left panel) and ATP (right panel) under the influence of various doses of heparin. As shown in FIG. 1C, autoradiography revealed that both GTP and ATP served as dependable sources to provide phosphoryl group to phosphorylate C in CK2-mediated kinase assay. Further, this reaction was severely inhibited by the CK2 inhibitor, heparin. The ability of CK2 to phosphorylate C was adversely affected by heparin at concentrations above 10 uM.

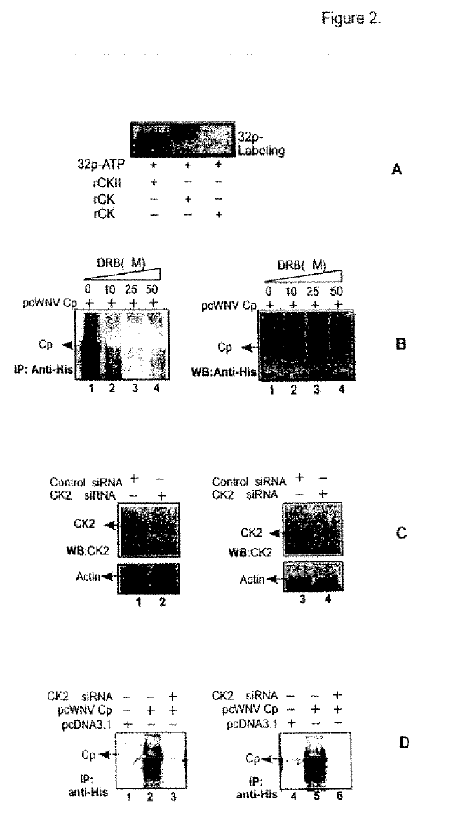

Neither CK2α nor CK2β alone phosphorylates WNV C. Human CK2 is constituted by CK2α and CK2β subunits. CK2α mediates the phosphorylation event while CK2β governs the regulatory role. Thus both of these subunits are required for phosphorylation of the proteins targeted by CK2. However, in few cases, CK2α alone can mediate the phosphorylation event of the target protein. We sought to clarify whether capsid protein is phosphorylated by CK2 a itself or by the holo enzyme complex consisting of CK2 α and CK2 β subunits. To address this question, the kinase assays were carried out using recombinant alpha and beta subunits using rC as the substrate. Neither of the subunits was able to phosphorylate the protein by themselves. However, when both these proteins are constituted together, they were able to transfer the radioactive phosphate to the capsid. Thus, in the case of WNV capsid, both alpha and beta subunits are required to phosphorylate capsid (FIG. 2A).

The in vivo phosphorylation of WNV C is inhibited by 5,6-Dichloro-1-(b-D-Ribofuranosyl) Bensimidazole, (DRB) a cell-permeable inhibitor of CK2. Subsequent to the identification and characterization of CK2-mediated phosphorylation of the rC in vitro, we wanted to study this phenomenon in vivo. In order to verify the CK2-mediated phosphorylation, DRB was examined as a specific inhibitor for CK2 activity. Unlike heparin, DRB is a cell-permeable compound and has been reported to specifically inhibit host cellular CK2 activity. Vero cells were transfected with pcWNV C-His expression construct that encodes capsid protein fused to poly histidine tag. The transfected cells were metabolically labeled with ³²P-phosphoric acid and incubated in the presence of the treatment with DRB. Following the treatment and lysis, the radio-labeled lysates were immunoprecipitated using anti-his antibody. As shown in FIG. 2B, the immunoprecipitation of radio-labeled capsid reveals that capsid is phosphorylated in vivo also. Further, gradual disappearance of radiolabeled capsid protein in the DRB-treated samples indicate that phosphorylation of capsid was severely inhibited by this CK2 antagonist. The lysates from the matching controls that were not exposed to radioactivity, were analyzed separately for capsid expression. An uniform level of capsid expression was observed (data not shown). The result strongly suggested that WNV C was phosphorylated by CK2 during the expression of capsid in human cells. A control without DRB showed that the addition of DMSO alone did not affect the phosphorylation of capsid (data not shown).

CK2α and CK2-β specific siRNA molecules inhibit the phosphorylation of capsid in vivo. To advance in our understanding of the phosphorylation phenomenon of WNV capsid in vivo, we utilized siRNA molecules that specifically inhibit CK2α and CK2β expression. Prior to their application, the ability of these molecules to selectively inhibit the expression of CK2α and CK2β subunits were verified in cell culture. 293 cells were transfected with siRNA-CK2α or siRNA-CK2β or siRNA-negative control that was prepared from a sequence that is not homologous to any known human gene sequences (provided by the supplier). The lysates from the transfected cells were analyzed by western blotting analysis using anti-CK2α/CK2β antibodies. FIG. 2C demonstrates that the addition of siRNA-CK2α to the cells significantly reduced the expression of CK2α production whereas siRNA-CK2β completely blocked the expression of CK2β as revealed by the Western analysis. Next, the cells were transfected with capsid-expression plasmids with siRNA-CK2α or siRNA-CK2β molecules. These transfected cells were pulse chased with ³²P-labeled orthophosphate and the lysates were subsequently verified to determine the extent of capsid phosphorylation in vivo as described earlier. Autoradiographic analysis confirmed the phosphorylation of the capsid protein from the lysates of capsid-transfected cells that were not treated with siRNA. The inhibition of the expression of CK2 subunits significantly impaired the in vivo phosphorylation of capsid in the transfected cells. The data in FIG. 2D, suggests successful incorporation of radioactive phosphoryl moiety only into protein C encoded by pcWNV C-His. Addition of siRNA-CK2α or siRNA-CK2β to the pcWNV C-transfected cells inhibited phosphorylation of capsid. Immunoprecipitation of lysates from pcDNA3.1-transfected cells using anti-His antibody did not yield a signal that corresponds to capsid from adjacent lanes. In sum, these results support that inhibition of either CK2α or CK2β clearly affects the phosphorylation of capsid in vivo.

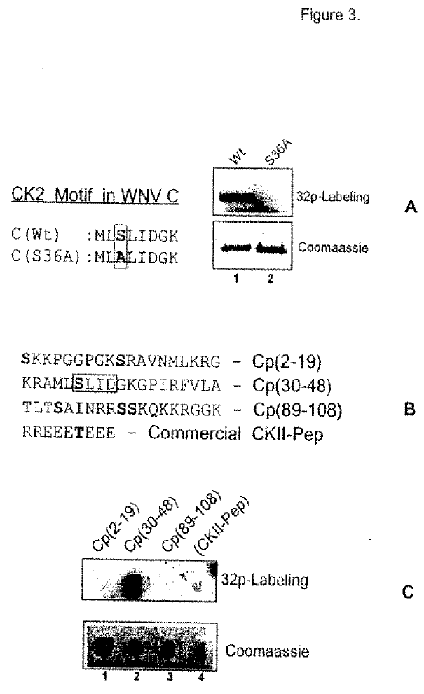

Ser-36 is the Major Site of CK2 Phosphorylation of WNV C. Subsequent to the identification and characterization of the components required for the phosphorylation of capsid, we wanted to identify the residue(s) that are phosphorylated by CK2. An analysis of deduced amino acid sequence at the ExPASy molecular biology server of the Swiss Institute of Bioinformatics (http://www.expasy.ch) identified a putative CK2 phosphorylation site. Amino acid sequence deduced from WNV C contained a sequence (34-SLID-37) (FIG. 3A) containing one Ser residue followed by xxD residues available for possible CK2 phosphorylation. This region agrees with the consensus CK2 motif (S/T)xx(D/E) (reviewed by Meggio and Pinna, 2003). To confirm the critical role of this motif in the event of phosphorylation by CK2, a capsid mutant in which, an Alanine residue substituted serine at 36, was constructed by overlap PCR [referred as rC(S36A)]. rC(S36A) mutant capsid protein was also bacterially expressed as described in Material and Methods and subjected to the CK2-kinase assay. As revealed in FIG. 3A, the phosphorylation signal was totally absent from C(S36A) while the wild rC still was labeled in the in vitro assay. Thus, the mutant capsid rC(S36A) failed to be phosphorylated by CK2 confirming the importance of this site. We examined whether the impairment of this mutant in its phosphorylation ability is related to any potential conformational changes that might have been caused by this mutation. To address this question, three different peptides representing different domains of capsid were prepared. Importantly, these peptides included serine residues in different locations. Only the peptide C(30-48) covered the (S/T)XX(D/E) motif, whereas other two peptides such as C(2-19) and C(89-108) included multiple serine residues. (FIG. 3B) These peptides were loaded onto nylon membrane (Applied Biosystems) using dot blot apparatus and the blots were subjected to kinase assay. Only the peptide C(30-48) yielded a strong signal for the uptake of ³²P and the extent of phosphorylation signal was greater than the positive control peptide provided by the commercial supplier. Whereas the other two peptides such as C(2-19) and C(89-108) turned to be negative in the assay even though multiple serine residues (FIG. 3C) occur both of these peptides. These results support that the phosphorylation of capsid is dependent on the Ser-36 residue and that CK2 phosphorylates serine only when it is located within the consensus motif that qualifies as a CK2 site. This observation concurs with the regulation of CK2-mediated phosphorylation in the mammalian system.

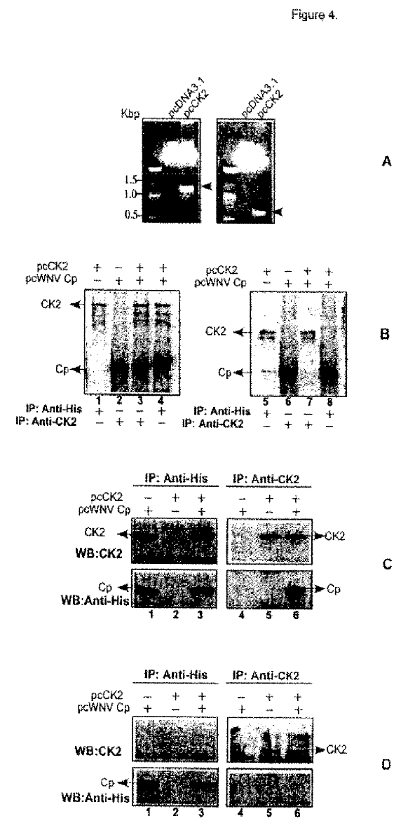

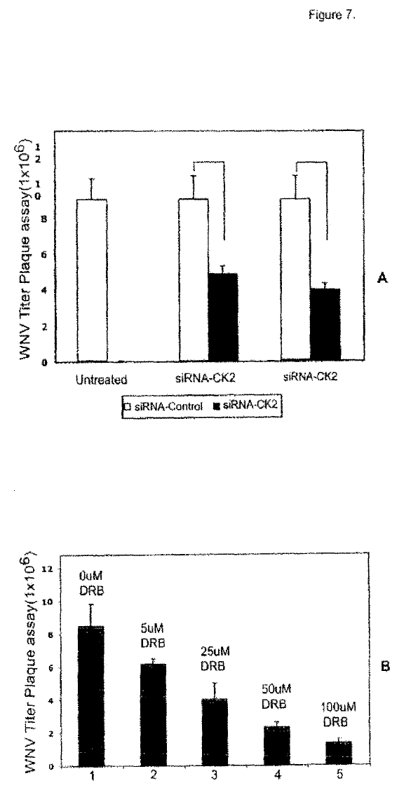

Capsid physically associates with CK2α and not with CK2β. Several reports described a physical interaction between various kinases and their target proteins. In this study, we were interested to examine whether capsid protein interacted physically with the CK2 complex. To address this question, first, human cDNAs encoding CK2 subunits, such as CK2α and CK2β, were cloned from total HeLa cDNA (FIG. 4A). The resulting constructs, pcCK2α and pcCK2β were used as templates to generate ³⁵S-labeled in vitro translated proteins samples. Radioactive capsid protein samples were prepared from pcWNV C-His. In vitro generated radio labeled protein samples were used to detect protein-protein interaction in a binding assay as described in materials and methods. In FIG. 7B, lane 1 indicates the protein product of about 63 kD derived from pcCK2α and the mass of this protein corresponded to the length of its open reading frame. pcWNV C generates a protein of about 14 kD. Capsid protein was incubated with the samples of either CK2α or CK2β. The resulting complexes were immunoprecipitated either with anti-His or anti-CK2α or anti-CK2β antibodies. When the mixture containing capsid and CK2α was cross immunoprecipitated, anti-His as well as anti-CK2α could co-immunoprecipitate both of these proteins. In the same way, the interaction between C and CK2β was examined. Immunoprecipitation of a gene product encoded by pcCK2β generated a product of about 29 kD that corresponds to its protein size (lane 5). Both CK2β and C radioactive samples were subjected to the binding assay and immunoprecipitated either by anti-His or anti-CK2β antibodies. Interestingly these two antibodies failed to immunoprecipitate both of the radio-labeled proteins. From the mix of radiolabeled C and CK2β samples, anti-His antibody was able to pull down only C whereas anti-CK2β could precipitate only CK2β, suggesting no direct interaction (FIG. 4B) between these two protein molecules in the transfected cells.

Subsequent to the results from in vitro binding assay, potential physical association between C and CK2 subunits was further examined in vivo also by western analysis. 293T cells were transfected with either capsid or CK2α alone or in combination of two plasmids. Two-days post transfection, the cells were lysed and the lysates were immunoprecipitated with anti-His antibody. The immunoprecipitated complexes were resolved by SDS-PAGE and immunoprobed by anti-CK2α sera. FIG. 4C lane 1 indicates the presence of CK2α pulled down by capsid from the lysates that were transfected with capsid alone. It is important that the presence of capsid in this lysate was sufficient to co-immunoprecipitate the indigenous level of CK2α. The signal for CK2α is absent in the lane 2 that contained only the CK2α construct and not capsid. In these cells, addition of anti-His targeting capsid, was unable to pull down CK2α. In the third lane that represents the lysate from the cells transfected with both capsid and CK2α plasmids, again anti-H is antibody was able to precipitate both capsid and CK2α. The bottom panel presents the expression of the C from the lanes wherever pcWNV C-His was included. The same lysates were immunoprecipitated by anti-CK2α followed by immunoprobing with anti-His antibody. Anti-CK2α could co-immunoprecipitate capsid when the cells were transfected with both pcCK2α and pcWNV C-His constructs. Thus, in line with the in vitro binding assay, western blot analysis of transfected cells also indicates a strong interaction between CK2α and WNV C in human cells.

Similarly, the association between CK2β and C in vivo was verified by western analysis. 293T cells were transfected with either capsid or CK2β construct alone or in combination of two plasmids. The resulting lysates were immunoprecipitated with anti-His or anti CK2β antibody. The immunoprecipitated complexes were resolved by SDS-PAGE and immunoprobed against anti-CK2β sera. In FIG. 4D, anti-His antibody could precipitate only the capsid protein, but failed to co-precipitate CK2β (FIG. 4D; panels 1-3). In the same way, anti-CK2β precipitated its antigen only, but not the capsid protein (FIG. 4D; panels 4-6). Thus, the physical association between CK2β and WNV C is not likely to occur. In sum, in concurrence with in vitro binding assay results, this co-immunoprecipitation followed by western analysis reveals that the capsid protein associates in vivo only with the CK2α and not with CK2β.

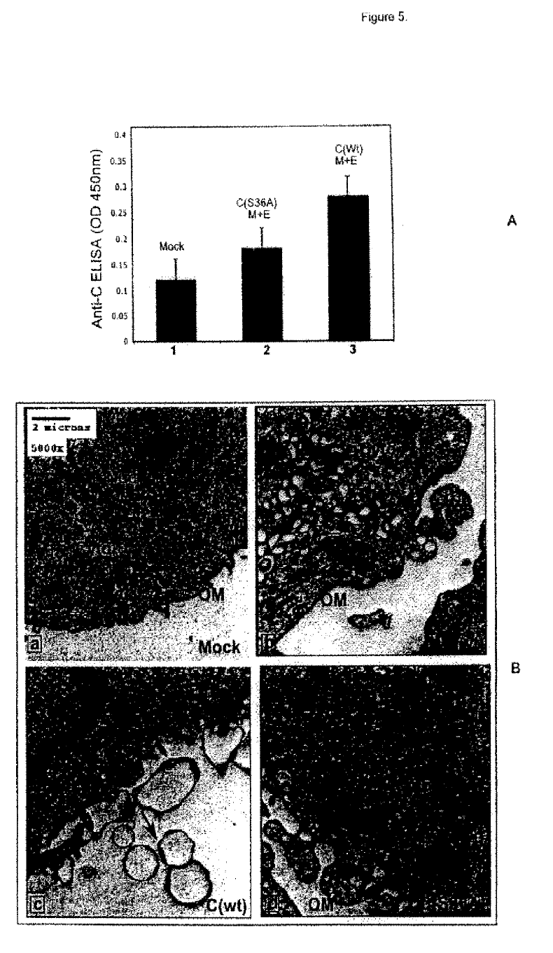

Phosphorylation of capsid is critical for the incorporation of capsid in VLPs. We examined whether the phosphorylation status of the protein C exerts any effect on capsid's biology. The mechanism(s) of the integration of capsid protein into the virus particles remains unclear. It has been demonstrated that the transfected cells expressing flaviviral preM and E alone, release viral like particles (VLPs) from their outer membrane. We have demonstrated that the cells expressing WNV C, along with E and preM also release capsid-containing virus-like-particles that resemble vesicular structures resembling VLPs as reported by Khromyyox et al (2004). In this study, we compared the efficiency of the capsid mutant, C(S36A) with the wild capsid protein, in participating into viral like particles. FIG. 5 reveals the evaluation of capsid release from the cells co-transfected with preM and E along with either C(wt) or C(S36A) or empty vector pcDNA3. Highest amount of capsid release was noticed from the cells expressing wild capsid protein. The inclusion of capsid-construct that encodes S36A mutant significantly reduced the release of capsid containing VLPs to the level equivalent to that of mock-transfected controls. Thus, the capsid mutant C(S36A) is significantly impaired with its ability to get incorporated into VLPs. The transfected cells were also analyzed by electron microscopy to visualize formation of these vesicular structures from cell surface. The surface of the mock-transfected cells is smooth as that of untransfected controls. However, as shown in FIG. 5B (b and c) from the cells that were transfected with C(wt)-expressing constructs, capsid-containing VLPs are released profoundly. In these cells the accumulation of these structures are in higher quantities are visible compared to that of the cells that express mutant capsid instead of the wild construct. Consistent with the ELISA data as in FIG. 5A, on the release of capsid-protein into the culture fluid, EM-analysis confirms that the mutant S36A mutant is impaired with its ability to participate in VLP production. Thus phosphorylation of serine-36 in capsid is modulating the participation of the capsid in viral formation through a mechanism that is yet to be ascertained yet.

CK2-specific inhibitor reduces the participation of capsid in VLPs. In consistent with the observation that mutant capsid is less efficient in getting incorporated into VLPs because of impaired ability of being phosphorylated, we sought to examine whether CK2 inhibitor exerts a similar effect. It is therefore presumable that, by inhibition of the activity of host cell CK2, participation of capsid in virus assembly can be significantly affected. Hence, we examined whether a cell-permeable CK2-specific inhibitor (DRB) exerts any effect on the efficiency of capsid in its participation in viral budding. The secretion of capsid containing VLPs was examined under the influence of DRB by ELISA measuring the level of capsid from the culture fluid. As shown in the FIG. 5C, the addition of DRB to the culture medium significantly reduced the level of capsid from the culture fluid. The level of inhibition appeared to be dose dependent also. These results support that DRB interferes with the ability of capsid protein to participate in viral budding by inhibiting the action of a cellular CK2 kinase that phosphorylates capsid. In sum, this data and the mutant data in FIGS. 5A and B support an important role of CK2 activity in phosphorylation of capsid for its participation in viral budding. Thus, the results of this study support that DRB specifically blocks the phosphorylation of capsid in both in vitro and in vivo assays

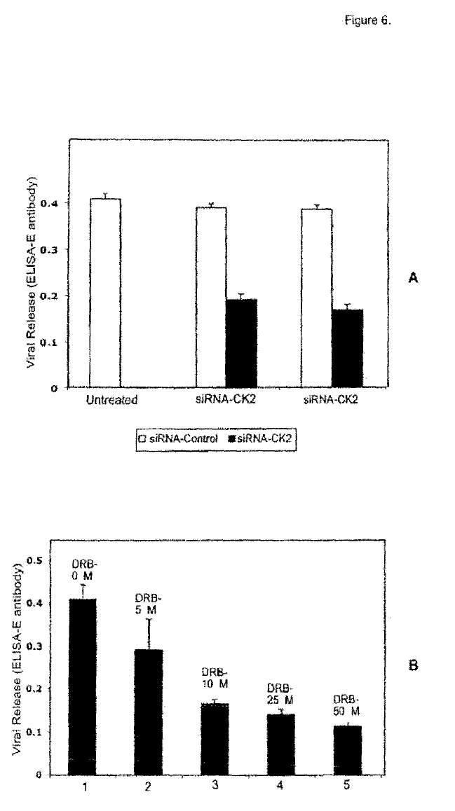

Both DRB and siRNA-CK2 molecules reduce WNV release. Following the identification of the effects of DRB and siRNA-CK2 molecules on the phosphorylation of capsid and its subsequent involvement in viral budding, we sought to extend this finding to the studies that utilized an infectious clone of WNV. In the context of whole virus, we examined whether inhibition of cellular CK2 affects WN viral release. We generated high titer of viral sample from Vero cells transfected with full length infectious clone (Pierson et al 2005). We harvested culture fluid at regular intervals, determined viral titre by routine Plaque assays and used them for infection assays. In the first approach, 293 cells were infected with a infectious WNV sup at 4 MOI under the treatment of either DRB or corresponding amount of solvent. Forty-eight hours post infection, viral release was monitored by both plaque assays as well as ELISAs measuring the presence of E antibody using monoclonal anti E antibody. FIG. 6B depicts the release of viral molecules from WNV-infected cells as measured by ELISA using anti-E antibody. Even at 5 uM, the effect of DRB on viral release was significant in comparison with the plates that were not treated with this inhibitor. However, at 10 uM, viral release was reduced by almost one fold and at 50 uM the effect of DRB was completely effective at blocking viral release. Therefore impairment of CK2 activity of the hosts cell by the addition of DRB reduced the level of viral release. While the inhibition of cellular CK2 activity results in the reduced viral load, it is presumable that depletion of CK2 transcripts also could cause similar effect. In order to test this, in the second approach, WNV release was evaluated under the conditions where expression of CK2 subunits was knocked out. Cellular CK2 was knocked out by using siRNA molecules that specifically target CK2α and CK2β transcripts. siRNA-CK2α and siRNA-CK2β molecules were used to deplete cellular transcripts of CK2. 293 cells were infected with infectious WNV followed by the treatment with either siRNA control molecules or siRNA-CK2α or siRNA-CK2β molecules. Release of WNV was monitored from these plates. As shown in FIG. 6A, siRNA-mock control did not affect the viral release whereas siRNA-CK2α reduced the viral release by one fold. In the same way, siRNA-CK2β also impaired the ability of viral release in comparison with the negative control. We also observed severe impairment of capsid phosphorylation in the pulse-chase studies under the influence of siRNA molecules. Thus in line with cell-culture studies as well as in vitro kinase assays, these studies suggest that loss of cellular CK2 activity by siRNA-molecules that target precisely the expression of CK2 kinase subunits leads to an adverse effect on WNV release. FIG. 7 presents the values of the viral titres from these studies involving siRNA-CK2 as well as DRB as determined by plaque assays that reveal the release of infectious WNV particles from these studies.

### Discussion

According to the invention, phosphorylation of WNV capsid protein is critical to viral replication and the mechanism underlying this process utilized host cellular kinase CFK2. The physiological relevance of this phosphorylation event is critical. Gel analysis of purified rC protein reveals the dimeric as well as monomeric nature of the protein. The multimeric nature of WNV capsid is consistent with of reports from Kunjin- (an Australian subtype of WNV), Tick-borne mosaic- and Dengue viruses. Capsid protein of these flaviviruses has been shown to organize into tetramers with highly positively charged surfaces.

Originally in was hypothesized that C-preM cleavage by host signalases results in the formation of membrane-associated “Cint” which is further processed by the NS2B-NS3 protease with formation of Cvir. A general scheme of flavivirus polyprotein processing has been proposed earlier: signalase cleavage of the C-preM precursor would result in formation of C retaining a stretch of hydrophobic amino acids which served as an internal signal sequence for the preM protein. A hydrophobic sequence at the carboxyl terminus of the C is absent in the mature capsid proteins but a variant with lower electrophoresis mobility had been reported in infected cells by few investigators. These findings led to the suggestion that the membrane-associated form of C, which resulted from signalase cleavage is further processed by a protease (complex) of viral origin to its mature form found in virions, and this cleavage is even connected with virion formation. Thus the existence of two forms capsid molecules is evident in WNV infected cells, though their biological as well as pathological relevance had been addressed individually.

The phosphorylation aspects of WNV C agree with the CK2-phosphorylation apparatus. The phosphorylation of C in vitro was severely impaired in a dose dependent manner by well-known CK2-specific inhibitors, such as heparin and DRB. These CK2-specific inhibitors have also been shown to inhibit the phosphorylation of other viral target proteins. CK2 is unique from other kinases that CK2 alone can recruit ATP as well as GTP as phosphate donors to transfer phosphoryl moieties to its target proteins. In this study, capsid was efficiently phosphorylated by CK2 using radioactive ATP as well as GTP with equal efficiency. Concurring with the in vitro results, the pulse chase experiments using ³²P-labeled orthophosphate also confirmed the phosphorylation of capsid cell-culture system also. In vivo phosphorylation of capsid is adversely inhibited by the cell-permeable inhibitor of CK2, DRB the phosphorylation of capsid protein in vivo. These results show that only CK2 is involved in the phosphorylation of this viral antigen.

Subsequent to the identification of CK2 as a key regulator of the phosphorylation of C, we further verified whether CK2α subunit alone mediates the transfer of the phosphoryl moiety. Using purified CK2 subunits, CK2α and CK2β proteins were tested for their ability to phosphorylate C. Neither of them were able phosphorylate C when they were provided individually. When both were constituted together, their phosphorylation efficacy was regained. There are few reports where protein targets are phosphorylated by CK2α alone. In the case of WNV C, it is evident that successful phosphorylation of this core protein depends on the integrity of the holoenzyme that includes both CK2α and CK2β. Available literature indicates that CK2α and α′ subunits, though structurally similar, are not functionally identical in mammals and yeast. Further, the holoenzyme may form transiently and dissociate in vivo. CKα is capable of nucleo-cytoplasmic shuttling and changes its sub cellular distribution. Free populations of α and β sub units exist alone or in association with different partners and there is increasing evidence that they may have specific functions. Thus, the distribution pattern of CK2 and its subunits, likely yields a diversified view. The holoenzyme and/or and its subunits are distributed throughout the cell and spreading from the outer membrane to the nucleolus.

Besides the dynamic state of CK2, the subunits of this kinase have been shown to interact with various cellular components. Our experimental results clearly suggest that CK2 is interacted by capsid protein. Whether dynamic distribution pattern of CK2 has any implication on the phosphorylation pattern of capsid and thereby in the pathological virulence of WNV is yet to be realized. Many viral proteins have been described as substrates for CK2 including the HSV-1 structural proteins VP22 and VP16. Although CK2 has been considered to be constitutively active, stimulation of its activity by stress signaling agents and heat shock factor can occur, while other agents inhibit its activity.

In order to phosphorylate C, it is presumed that CK2 and the capsid protein must be in a close vicinity with or without physical interaction between the catalytic kinase unit and its target protein. In vitro protein binding assays using ³⁵S-labeled samples as well as Western blotting analysis indicated that the alpha subunit of CK2 physically interacts with C. Though several viral proteins have been shown to modulate/activate and redistribute CK2 sub units in the cell, only a few of them have been shown to interact with CK2 physically so far. Herpes Simplex Virus ICP27 protein is one such a protein shown to interact physically with CK2 in infected cells.

Besides the CK2-specific inhibitors, siRNA-CKα and siRNA-CK2β also specifically impaired the ability of the capsid protein in getting phosphorylated in the transfected cells. This yields a strong evidence that the WNV core protein is phosphorylated only by host cellular CK2 and none by other kinases though capsid still harbors a phosphorylation site for protein kinase C. If PKC is involved in phosphorylation of C, DRB as well as siRNA-CK2 molecules should have not affected the phosphorylation of capsid. Rather these reagents clearly inhibited the phosphorylation event in the cell-culture studies and thus establishing that CK2 is the sole candidate kinase that phosphorylates capsid.

In WNV C, we demonstrated that Serine-36 within the conserved CK2-motif is the target residue to be phosphorylated by CK2. As anticipated, the capsid mutant, C36A is not phosphorylated by CK2 in both in vitro assays and cell culture studies. Additional support to the essential role of this consensus CK2 motif in the phosphorylation of capsid by CK2 is available from a dot-blot analysis using peptides derived from capsid. The capsid peptides representing three different regions encompassing serine and threonine residues were used as the substrate for an in vitro CK2 kinase activity. Only the peptide containing the conserved CK2 motif was phosphorylated while the other two peptides turned negative for the incorporation of radioactive phosphate. Besides confirming the specificity of the kinase assays, this dot-blot kinase assay also suggests that any potential conformational changes in the capsid protein may not affect its phosphorylation by CK2. A recent structural analysis involving a pure protein from Kunjin virus indicates that this particular region also represents an alpha helical region. It is presumable that WNV capsid will also have similar structural features, as Kunjin virus capsid is homologous to WNV C except for two substitutions. However it is unclear whether phosphorylation of Serine-38 exerts any topological effect on this alpha-sheet in WNV capsid.

While the experimental results suggest the protein kinase CK2 as a sole mediator of the phosphorylation of capsid, the biological relevance of CK2 deserves special consideration. CK2 involved in the phosphorylation of several viral antigens. Also, in some cases, viral infections resulted in the upregulation CK2 activity as well as redistribution of CK2 elements throughout the cytoplasm. Thus it is tempting to suggest that phosphorylation of the capsid protein by CK2 may exert significant effects on certain stages of the viral cycle that depend on the inclusion of the capsid. Available literature suggests the formation of subviral particles during viral replication just from the assembly of membrane and E proteins themselves. However, an infection particle could be formed only upon the inclusion of capsid into the virion. Probably, the phosphorylation status of capsid may be critical in the events leading to the participation of capsid in the virion formation. This is in turn supported by the observed reduction in capsid protein in the cell culture fluid of the cells transfected with the mutant capsid.

Whether WNV infection modulates CK2 activities in the infected cells either by activating their redistribution pattern or by directly interacting with them remains to be resolved. We show that phosphorylation of CK2-motif of the capsid is critical for its involvement in the viral assembly. We sought to address whether phosphorylation of capsid has any role in the incorporation of capsid in viral particles. In the transfection system, complementing the mutant capsid significantly reduced the incorporation of capsid. Thus, Serine-36 has a role in the incorporation of capsid in viral assembly. The explanation on the role of this residue in affecting the incorporation of capsid in VLPs is yet to be arrived. Subsequent to this observation, the virus bearing Alanine substitution in the place of Serine-36 has been observed to have a reduced level of budding. In line with this observation, DRB as well as CK2-siRNA molecules also significantly reduced the viral release.

Though the mechanism of virus replication has been understood to certain level, the information on capsid-RNA interaction, capsid-E interaction and other roles of capsid are yet to be resolved. Understanding the precise involvement of host-factors in governing WNV assembly and budding from infected cells requires thorough investigations. Involvement of cellular kinases in any of these steps of viral replication will add a new dimension to our understanding of WNV biology and yields potential avenues for designing therapeutic strategies against WNV infection. Experimental results of this study leave several questions open to the readers. (a) How the phosphorylation of serine-36, modulates the incorporation of capsid in viral assembly, (b) If capsid-genomic RNA interaction is dependent on the phosphorylation status of capsid by CK2, then CK2 may potentially influence the efficient participation of capsid in encapsulation step during WNV life cycle, and (c) WNV assembly/budding may involve steps that are energy dependent and mediated by versatile CK2. Capsid is phosphorylable from both ATP and GTP as donors and hence indicates the involvement of ATPases and GTPases, the role of which are demonstrated in other models of viral replication. In this scenario, inhibition of CK2 activity by CK2-specific inhibitors may hamper the participation of capsid. We have shown the selective inhibition of CK2 by DRB that is fatal to CK2 activity and siRNA-CK2 molecules that inhibits the expression of this protein kinase. Thus precise interference of cellular CK2 activity by using inhibitors such as DRB and the expression level of CK2 subunits by employing siRNA-CK2 molecules offers valuable tools in the development of therapeutic strategies in treating WNV infection.

### REFERENCES

1. Akten, B., E. Jauch, G. K. Genova, E. Y. Kim, I. Edery, T. Raabe, and F. R. Jackson.
2003. A role for CK2 in the Drosophila circadian oscillator. Nat Neurosci 6:251-7.
2. Anderson, J. F., T. G. Andreadis, C. R. Vossbrinck, S. Tirrell, E. M. Wakem, R. A. French, A. E. Garmendia, and H. J. Van Kruiningen. 1999. Isolation of West Nile virus from mosquitoes, crows, and a Cooper's hawk in Connecticut. Science 286:2331-3.
3. Batik, S., and A. K. Banerjee. 1992. Phosphorylation by cellular casein kinase II is essential for transcriptional activity of vesicular stomatitis virus phosphoprotein P. Proc Natl Acad Sci USA 89:6570-4.
4. Batik, S., and A. K. Banerjee. 1992. Sequential phosphorylation of the phosphoprotein of vesicular stomatitis virus by cellular and viral protein kinases is essential for transcription activation. J Virol 66:1109-18.
5. Barrasa, M. I., N. Y. Harel, and J. C. Alwine. 2005. The Phosphorylation Status of the Serine-Rich Region of the Human Cytomegalovirus 86-Kilodalton Major Immediate-Early Protein IE2/IEP86 Affects Temporal Viral Gene Expression. J Virol 79:1428-37.
6. Brinton, M. A. 2002. The molecular biology of West Nile Virus: a new invader of the western hemisphere. Annu Rev Microbio156:371-402.
7. Cheng, Y., N. J. King, and A. M. Kesson. 2004. Major histocompatibility complex class I (MHC-I) induction by West Nile virus: involvement of 2 signaling pathways in MHC-1 up-regulation. J Infect Dis 189:658-68.
8. Chu, J. J., and M. L. Ng. 2003. The mechanism of cell death during West Nile virus infection is dependent on initial infectious dose. J Gen Virol 84:3305-14.
9. Daniel, X., S. Sugano, and E. M. Tobin. 2004. CK2 phosphorylation of CCA1 is necessary for its circadian oscillator function in Arabidopsis. Proc Natl Acad Sci USA 101:3292-7.
10. Dotan, I., E. Ziv, N. Dafni, J. S. Beckman, R. O. McCann, C. V. Glover, and D. Canaani.
2001. Functional conservation between the human, nematode, and yeast CK2 cell cycle genes. Biochem Biophys Res Commun 288:603-9.
11. E1-Guindy, A. S., and G. Miller. 2004. Phosphorylation of Epstein-Barr virus ZEBRA protein at its casein kinase 2 sites mediates its ability to repress activation of a viral lytic cycle late gene by Rta. J Virol 78:7634-44.
12. Filhol, O., J.-L. Martiel, and C. Cochet. 2004. Protein kinase CK2: a new view of an old molecular complex. EMBO Rep 5:351-355.
13. Hu, P., K. Samudre, S. Wu, Y. Sun, and N. Hernandez. 2004. CK2 phosphorylation of Bdp1 executes cell cycle-specific RNA polymerase III transcription repression. Mol Cell 16:81-92.
14. Hu, P., S. Wu, and N. Hernandez. 2003. A minimal RNA polymerase III transcription system from human cells reveals positive and negative regulatory roles for CK2. Mol Cell 12:699-709.
15. Hui, E. K., and D. P. Nayak. 2002. Role of G protein and protein kinase signaling in influenza virus budding in MDCK cells. J Gen Virol 83:3055-66.
16. Ivanov, K. I., P. Puustinen, R. Gabrenaite, H. Vihinen, L. Ronnstrand, L. Valmu, N. Kalkkinen, and K. Makinen. 2003. Phosphorylation of the potyvirus capsid protein by protein kinase CK2 and its relevance for virus infection. Plant Cell 15:2124-39.
17. Jones, T. C., L. Ma, J. W. Burgner, T. D. Groesch, B. C. Post, and R. J. Kuhn. 2003. Flavivirus capsid is a dimeric alpha-helical protein. J Virol 77:7143-49.
18. Kaushik, R., and M. S. Shaila. 2004. Cellular casein kinase II-mediated phosphorylation of rinderpest virus P protein is a prerequisite for its role in replication/transcription of the genome. J Gen Virol 85:687-91.
19. Kesson, A. M., Y. Cheng, and N. J. King. 2002. Regulation of immune recognition molecules by flavivirus, West Nile. Viral Immunol 15:273-83.
20. Kesson, A. M., and N. J. King. 2001. Transcriptional regulation of major histocompatibility complex class I by flavivirus West Nile is dependent on NF-kappaB activation. J Infect Dis 184:947-54.
21. Khromykh, A. A., and E. G. Westaway. 1996. RNA binding properties of core protein of the flavivirus Kunjin. Arch Virol 141:685-99.
22. Kiermayr, S., R. M. Kofler, C. W. Mandl, P. Messner, and F. X. Heinz. 2004. Isolation of capsid protein dimers from the Tick-Borne Encephalitis Flavivirus and in vitro assembly of capsid-like particles. J Virol 78:8078-84.
23. Koffa, M. D., J. Kean, G. Zachos, S. A. Rice, and J. B. Clements. 2003. CK2 protein kinase is stimulated and redistributed by functional herpes simplex virus ICP27 protein. J Virol 77:4315-25.
24. Kohl, A., V. di Bartolo, and M. Bouloy. 1999. The Rift Valley fever virus nonstructural protein NSs is phosphorylated at serine residues located in casein kinase II consensus motifs in the carboxy-terminus. Virology 263:517-25.
25. Lanciotti, R. S., J. T. Roehrig, V. Deubel, J. Smith, M. Parker, K. Steele, B. Crise, K. E. Volpe, M. B. Crabtree, J. H. Scherret, R. A. Hall, J. S. MacKenzie, C. B. Cropp, B. Panigrahy, E. Ostlund, B. Schmitt, M. Malkinson, C. Banet, J. Weissman, N. Komar, H. M. Savage, W. Stone, T. McNamara, and D. J. Gubler. 1999. Origin of the West Nile virus responsible for an outbreak of encephalitis in the northeastern United States. Science 286:2333-7.
26. Lindenbach, B. D., and C. M. Rice. 2001. Flaviviridae: the viruses and their replication, p. 991-1041. In D. M. Knipe, P. M. Howley, D. E. Griffin, R. A. Lamb, M. A. Martin, B. Roizman, and S. E. Straus (ed.), Fields Virology. Lippincott, Williams & Wilkins., Philadelphia, Pa.
27. Lorenz, P., R. Pepperkok, and W. Pyerin. 1994. Requirement of casein kinase 2 for entry into and progression through early phases of the cell cycle. Cell Mol Biol Res 40:519-27.
28. Malik, P., and J. B. Clements. 2004. Protein kinase CK2 phosphorylation regulates the interaction of Kaposi's sarcoma-associated herpesvirus regulatory protein ORF57 with its multifunctional partner hnRNP K. Nucl Acids Res 32:5553-5569.
29. Meggio, F., D. M. D′Agostino, V. Ciminale, L. Chieco-Bianchi, and L. A. Pinna. 1996. Phosphorylation of HIV-1 Rev protein: implication of protein kinase CK2 and pro-directed kinases. Biochem Biophys Res Commun 226:547-54.
30. Meggio, F., O. Marin, M. Boschetti, S. Sarno, and L. A. Pinna. 2001. HIV-1 Rev transactivator: a beta-subunit directed substrate and effector of protein kinase CK2. Mol Cell Biochem 227:145-51.
31. Meggio, F., and L. A. Pinna. 2003. One-thousand-and-one substrates of protein kinase CK2? Faseb J 17:349-68.
32. O'Reilly, D., O. Hanscombe, and P. O'Hare. 1997. A single serine residue at position 375 of VP16 is critical for complex assembly with Oct-1 and HCF and is a target of phosphorylation by casein kinase II. Embo J 16:2420-30.
33. Parquet, M. C., A. Kumatori, F. Hasebe, K. Morita, and A. Igarashi. 2001. West Nile virus-induced bax-dependent apoptosis. FEBS Lett 500:17-24.
34. Petersen, L. R., A. Marfin, A., and D. J. Gubler. 2003. West Nile Virus. Jama 290:524-28.
35. Peterson, L. R., and J. T. Roehrig. 2001. West Nile Virus: a reemerging global pathogen. Emerging Infectous Diseases 7:611-614.
36. Pinna, L. A. 2002. Protein kinase CK2: a challenge to canons. J Cell Sci 115:3873-8.
37. Rice, C. M. 1996. Flaviviridae: The viruses and their replication, p. 940. In B. N. Fields, D. M. Knipe, and P. M. Howley (ed.), Virology, vol. 1. Raven Press, New York.
38. Roach, P. J. 1991. Multisite and hierarchal protein phosphorylation. J Biol Chem 266:14139-42.
39. Romieu-Mourez, R., E. Landesman-Bollag, D. C. Seldin, and G. E. Sonenshein. 2002. Protein kinase CK2 promotes aberrant activation of nuclear factor-kappaB, transformed phenotype, and survival of breast cancer cells. Cancer Res 62:6770-8.
40. Romieu-Mourez, R., E. Landesman-Bollag, D. C. Seldin, A. M. Traish, F. Mercurio, and G. E. Sonenshein. 2001. Roles of IKK kinases and protein kinase CK2 in activation of nuclear factor-kappaB in breast cancer. Cancer Res 61:3810-8.
41. Sayed, M., S. Pelech, C. Wong, A. Marotta, and B. Salh. 2001. Protein kinase CK2 is involved in G2 arrest and apoptosis following spindle damage in epithelial cells. Oncogene 20:6994-7005.
42. Shimada, K., K. Kondo, and K. Yamanishi. 2004. Human herpesvirus 6 immediate-early 2 protein interacts with heterogeneous ribonucleoprotein K and casein kinase 2. Microbiol Immunol 48:205-10.
43. Singh, D. K., D. M. Griffin, E. Pacyniak, M. Jackson, M. J. Werle, B. Wisdom, F. Sun, D. R. Hout, D. M. Pinson, R. S. Gunderson, M. F. Powers, S. W. Wong, and E. B. Stephens. 2003. The presence of the casein kinase II phosphorylation sites of Vpu enhances the CD4(+) T cell loss caused by the simian-human immunodeficiency virus SHIV(KU-lbMC33) in pig-tailed macaques. Virology 313:435-51.
44. Unger, G. M., A. T. Davis, J. W. Slaton, and K. Ahmed. 2004. Protein kinase CK2 as regulator of cell survival: implications for cancer therapy. Curr Cancer Drug Targets 4:77-84.
45. Wang, T., T. Town, L. Alexopoulou, J. F. Anderson, E. Fikrig, and R. A. Flavell. 2004. Toll-like receptor 3 mediates West Nile virus entry into the brain causing lethal encephalitis. Nat Med 10:1366-73.
46. Westaway, E. G., A. A. Khromykh, M. T. Kenney, J. M. Mackenzie, and M. K. Jones.
1997. Proteins C and NS4B of the flavivirus Kunjin translocate independently into the nucleus. Virol 234:31-41.
47. Wu, X., X. Lei, and Z. F. Fu. 2003. Rabies virus nucleoprotein is phosphorylated by cellular casein kinase II. Biochem Biophys Res Commun 304:333-8.
48. Xiao, S. Y., H. Guzman, H. Zhang, A. P. A. T. Rosa, and R. B. Tesh. 2001. West Nile virus infection in the golden hamster (Mesocricetus auratus): A model for West Nile encephalitis. Emerg Infect Dis 7:714-721.
49. Yang, J., S., M. P. Ramanathan, K. Muthumani, S. H. Jin, Q. C. Yu, A. Y. Choo, D. H. Hwang, M. D. Lee, K. Dang, W. Dang, J. J. Kim, and D. B. Weiner. 2002. Induction of inflammation by West Nile Virus Capsid through the Caspase-9 Apoptotic Pathway. Emer Infect Dis 8:1379-1384.
50. Perri R B, Almquist S J, Byrn R A, Chandorkar G, Chaturvedi P R, Courtney L F, Decker C J, Dinehart K, Gates C A, Harbeson S L, Heiser A, Kalkeri G, Kolaczkowski E, Lin K, Luong Y P, Rao B G, Taylor W P, Thomson J A, Tung R D, Wei Y, Kwong A D, Lin C. Preclinical profile of VX-950, a potent, selective, and orally bioavailable inhibitor of hepatitis C virus NS3-4A serine protease. Antimicrob Agents Chemother. 2006 March; 50(3):899-909.
51. Lin C, Kwong A D, Perni R B. Discovery and development of VX-950, a novel, covalent, and reversible inhibitor of hepatitis C virus NS3.4A serine protease. Infect Disord Drug Targets. 2006 March; 6(1):3-1
52. Discovery of (1R,5S)—N-[3-amino-1-(cyclobutylmethyl)-2,3-dioxopropyl]-3-[2(S)-[[[(1,1-dimethylethyl)amino]carbonyl]amino]-3,3-dimethyl-1-oxobutyl]-6,6-dimethyl-3-azabicyclo[3.1.0]hexan-2(S)-carboxamide (SCH 503034), a selective, potent, orally bioavailable hepatitis C virus NS3 protease inhibitor: a potential therapeutic agent for the treatment of hepatitis C infection. J Med. Chem. 2006 Oct. 5; 49(20):6074-86.
53. Franck N, Le Seyec J, Guguen-Guillouzo C, Erdtmann L., Hepatitis C virus NS2 protein is phosphorylated by the protein kinase CK2 and targeted for degradation to the proteasome. Virol. 2005 March; 79(5):2700-8.
54. Quintavalle M, Sambucini S, Di Pietro C, De Francesco R, Neddermann P. The {alpha}-isoform of protein kinase CKI is responsible for Hepatitis C Virus NS5A hyperphosphorylation. J. Virol. 2006 Aug. 30

1. A method of treating an individual infected with a virus selected from the group consisting of: West Nile Virus, Japanese encephalitis virus, Kunjin virus Tick-borne encephalitis virus and Hepatitis C virus, comprising administering to such individual, a therapeutically effective amount of one or more compounds that inhibit CK2 activity, one or more compounds that inhibit CK2 expression or a combination thereof.

2. The method of claim 1 comprising administering to such individual a compound that inhibits CK2 activity.

3. The method of claim 2 wherein the compound that inhibits CK2 activity is selected from the group consisting of: DRB, TBB; TBBt; DMAT; Myricetin; emodin; and aloe-emodin.

4. The method of claim 1 comprising administering to such individual a compound that inhibits CK2 expression.

5. The method of claim 4 wherein the compound that inhibits CK2 expression is selected from the group consisting of: siRNA oligonucleotides that inhibit expression of CK2α; siRNA oligonucleotides that inhibit expression of CK2α′; siRNA oligonucleotides that inhibit expression of CKβ; antisense oligonucleotides that inhibit expression of CK2α; antisense oligonucleotides that inhibit expression of CK2α′; and antisense oligonucleotides that inhibit expression of CKβ.

6. A method of treating an individual exposed to a virus selected from the group consisting of: West Nile Virus, Japanese encephalitis virus, Kunjin virus Tick-borne encephalitis virus and Hepatitis C virus, comprising administering to such individual, a therapeutically effective amount of one or more compounds that inhibit CK2 activity, one or more compounds that inhibit CK2 expression or a combination thereof.

7. The method of claim 6 comprising administering to such individual a compound that inhibits CK2 activity.

8. The method of claim 7 wherein the compound that inhibits CK2 activity is selected from the group consisting of: DRB, TBB; TBBt; DMAT; Myricetin; emodin; and aloe-emodin.

9. The method of claim 6 comprising administering to such individual a compound that inhibits CK2 expression.

10. The method of claim 9 wherein the compound that inhibits CK2 expression is selected from the group consisting of: siRNA oligonucleotides that inhibit expression of CK2α; siRNA oligonucleotides that inhibit expression of CK2α′; siRNA oligonucleotides that inhibit expression of CKβ; antisense oligonucleotides that inhibit expression of CK2α; antisense oligonucleotides that inhibit expression of CK2α′; and antisense oligonucleotides that inhibit expression of CKβ.

11. Pharmaceutical compositions comprising therapeutically effective amount of one or more compounds that inhibit CK2 activity, one or more compounds that inhibit CK2 expression, or a combination thereof.

12. The composition of claim 11 comprising a compound that inhibits CK2 activity selected from the group consisting of: DRB, TBB; TBBt; DMAT; Myricetin; emodin; and aloe-emodin.

13. The composition of claim 11 comprising a compound that inhibits CK2 expression selected from the group consisting of: siRNA oligonucleotides that inhibit expression of CK2α; siRNA oligonucleotides that inhibit expression of CK2α′; siRNA oligonucleotides that inhibit expression of CKβ; antisense oligonucleotides that inhibit expression of CK2α; antisense oligonucleotides that inhibit expression of CK2α′; and antisense oligonucleotides that inhibit expression of CKβ.

14. A method of inhibiting viral replication by a virus selected from the group consisting of: West Nile Virus, Japanese encephalitis virus, Kunjin virus Tick-borne encephalitis virus and Hepatitis C virus, comprising the step of: contacting an antiviral composition selected from the group consisting of: one or more compounds that inhibits CK2 activity, one or more compounds that inhibits CK2 expression and combinations thereof, with cells that are infected with a virus selected from group consisting of: West Nile Virus, Japanese encephalitis virus, Kunjin virus Tick-borne encephalitis virus and Hepatitis C virus under conditions in which viral replication occurs in the absence of the antiviral composition.

15. The method of claim 14 wherein the antiviral composition comprises a compound that inhibits CK2 activity.

16. The method of claim 15 wherein the compound that inhibits CK2 activity is selected from the group consisting of: DRB, TBB; TBBt; DMAT; Myricetin; emodin; and aloe-emodin.

17. The method of claim 14 wherein the antiviral composition comprises a compound that inhibits CK2 expression.

18. The method of claim 17 wherein the compound that inhibits CK2 expression is selected from the group consisting of: siRNA oligonucleotides that inhibit expression of CK2α; siRNA oligonucleotides that inhibit expression of CK2α′; siRNA oligonucleotides that inhibit expression of CKβ; antisense oligonucleotides that inhibit expression of CK2α; antisense oligonucleotides that inhibit expression of CK2α′; and antisense oligonucleotides that inhibit expression of CKβ.

19. A method of identifying a compound useful to treat infection by a virus selected from group consisting of: West Nile Virus, Japanese encephalitis virus, Kunjin virus Tick-borne encephalitis virus and Hepatitis C virus, comprising the steps of:
a) performing a test assay in which a test compound is contacted with CK2 in the presence of a substrate, in conditions under which said CK2 phosphorylates said substrate in the absence of said test compound and comparing the amount of phosphorylation observed in said test assay with the amount of phosphorylation that occurs when CK2 is contacted with substrate, in conditions under which said CK2 phosphorylates said substrate in the absence of said test compound; wherein a lower amount of phosphorylation observed in said test assay compared to the amount of phosphorylation that occurs in the absence of the test compound indicates that the test compound inhibits CK2 activity;
b) contacting the test compound that inhibits CK2 activity with cells that are infected with a virus selected from group consisting of: West Nile Virus, Japanese encephalitis virus, Kunjin virus Tick-borne encephalitis virus and Hepatitis C virus under conditions in which viral replication occurs in the absence of the test compound, and comparing the level of viral replication that occurs to the presence of the test compound with the level of viral replication that occurs to the absence of the test compound; wherein a reduction of the level of viral replication that occurs to the presence of the test compound compared to the level of viral replication that occurs to the presence of the test compound indicates that the compound is useful to treat infection by a virus selected from group consisting of: West Nile Virus, Japanese encephalitis virus, Kunjin virus Tick-borne encephalitis virus and Hepatitis C virus.

20. A method of identifying a compound that inhibits CK2 comprising the steps of:
performing a test assay in which a test compound is contacted with CK2 in the presence of a substrate, in conditions under which said CK2 phosphorylates said substrate in the absence of said test compound and comparing the amount of phosphorylation observed in said test assay with the amount of phosphorylation that occurs when CK2 is contacted with substrate, in conditions under which said CK2 phosphorylates said substrate in the absence of said test compound; wherein the substrate is selected from the group consisting of: West Nile Virus C protein, Japanese encephalitis virus C protein, Kunjin virus C protein, Tick-borne encephalitis virus C protein, and Hepatitis C virus NS2/NS3 protein, and wherein a lower amount of phosphorylation observed in said test assay compared to the amount of phosphorylation that occurs in the absence of the test compound indicates that the test compound inhibits CK2 activity.
